(19) United States
(12) Patent Application Publication
(10) Pub. No.: US 2009/0202921 A1
(43) Pub. Date: Aug. 13, 2009

(54) OPTICAL RECORDING COMPOSITION AND HOLOGRAPHIC RECORDING MEDIUM

(75) Inventors: Hiroyuki SUZUKI, Kanagawa (JP); Satoru Yamada, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/368,380

(22) Filed: Feb. 10, 2009

(30) Foreign Application Priority Data
Feb. 12, 2008 (JP) 2008-030184
Aug. 22, 2008 (JP) 2008-213533

(51) Int. Cl. G03F 7/00; C07D 277/64; C07D 263/56; C07D 235/16; C07D 405/12; C07D 209/18; C07D 417/12

(52) U.S. Cl. 430/2; 548/180; 548/217; 548/309.7; 548/304.7; 548/469; 548/156

(57) ABSTRACT

The present invention provides an optical recording composition comprising at least one compound denoted by general formula (I) and a holographic recording medium comprising a recording layer, wherein the recording layer comprises at least one compound denoted by general formula (I).

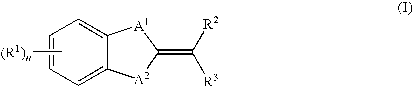

In general formula (I), each of A¹ and A² independently denotes —CR⁴R⁵—, —O—, —NR⁶—, —S—, or —C(═O)—, each of R⁴, R⁵, and R⁶ independently denotes a hydrogen atom or a substituent, R¹ denotes a substituent, n denotes an integer ranging from 0 to 4, each of R² and R³ independently denotes a substituent having a Hammett substituent constant, σp value, of greater than 0, R² and R³ do not form a ring structure by bonding together, and at least one from among R¹, R², R³, A¹, and A² comprises at least one polymerizable group.

### CROSS-REFERENCE TO RELATED APPLICATIONS

This application claims the benefit of priority under 35 USC 119 to Japanese Patent Application No. 2008-30184 filed on Feb. 12, 2008 and Japanese Patent Application No. 2008-213533 filed on Aug. 22, 2008, which are expressly incorporated herein by reference in their entirety.

### BACKGROUND OF THE INVENTION

1. Field of the Invention

The present invention relates to an optical recording composition comprising at least one optical recording compound in the form of a merocyanine compound, and more particularly, to an optical recording composition suited to the manufacturing of a holographic recording medium permitting the writing of information, for example, with a 405 nm laser, particularly a volume holographic recording medium having a relatively thick recording layer. The present invention further relates to a holographic recording medium comprising a recording layer comprising the above optical recording compound.

2. Discussion of the Background

Holographic optical recording media based on the principle of the holograph have been developed. Recording of information on holographic optical recording media is carried out by superposing an informing light containing image information and a reference light in a recording layer comprised of a photosensitive composition to write an interference fringe thus formed in the recording layer. During the reproduction of information, a reference light is directed at a prescribed angle into the recording layer in which the information has been recorded, causing optical diffraction of the reference light by the interference fringe which has been formed, reproducing the informing light. For example, Published Japanese Translation of PCT International Application (TOKUHYO) No. 2005-502918 or English language family member US 2003/0087104 A1, which are expressly incorporated herein by reference in their entirety, discloses the use of a urethane matrix and a phenyl acrylate derivative in a holographic recording medium of the photopolymer type.

In recent years, volume holography, and, more particularly, digital volume holography, have been developed to practical levels for ultrahigh-density optical recording and have been garnering attention. Volume holography is a method of writing interference fringes three-dimensionally by also actively utilizing the direction of thickness of an optical recording medium. It is advantageous in that increasing the thickness permits greater diffraction efficiency and multiplexed recording increases the recording capacity. Digital volume holography is a computer-oriented holographic recording method in which the image data being recorded are limited to a binary digital pattern while employing a recording medium and recording system similar to those of volume holography. In digital volume holography, for example, image information such as an analog drawing is first digitized and then expanded into two-dimensional digital pattern information, which is recorded as image information. During reproduction, the digital pattern information is read and decoded to restore the original image information, which is displayed. Thus, even when the signal-to-noise (S/N) ratio deteriorates somewhat during reproduction, by conducting differential detection or conducting error correction by encoding the two-dimensional data, it is possible to reproduce the original data in an extremely faithful manner (see Japanese Unexamined Patent Publication (KOKAI) Heisei No. 11-311936 or English language family member US 2002/0114027 A1, which are expressly incorporated herein by reference in their entirety).

Greater recording capacity is being demanded of the above volume holographic optical recording media. For example, Japanese Unexamined Patent Publication (KOKAI) No. 2005-275158, US 2005/233246 A1, and Japanese Unexamined Patent Publication (KOKAI) No. 2007-272044, which are expressly incorporated herein by reference in their entirety, disclose recording media comprising recording monomers in the form of dye compounds for the purpose of increasing recording capacity.

The wavelength of the recording light has tended to become shorter in recent years to increase recording capacity. Specifically, the use of 405 nm recording lights has begun. However, in the recording media described in Japanese Unexamined Patent Publication (KOKAI) No. 2005-275158, US 2005/233246 A1, and Japanese Unexamined Patent Publication (KOKAI) No. 2007-272044, the transmittance at wavelengths around 400 nm of the media has decreased due to the use of dye compounds having substantial absorption in the visible light region. Thus, the above media make it difficult to conduct high-sensitivity recording with a recording light having a wavelength around 400 nm.

### SUMMARY OF THE INVENTION

An aspect of the present invention provides for an optical recording composition comprising an optical recording compound suited to high-sensitivity digital volume holography with large storage capacity in recording with short-wavelength light, and for a holographic recording medium permitting ultrahigh-density optical recording using the above optical recording composition.

As the result of extensive research, the present inventors discovered that it was possible to achieve a holographic recording medium capable of high-density and high-sensitivity recording with short-wavelength light by using the merocyanine compound denoted by general formula (I) below. The present invention was devised on that basis.

An aspect of the present invention relates to an optical recording composition comprising at least one compound denoted by general formula (I).

A further aspect of the present invention relates to a holographic recording medium comprising a recording layer, wherein the recording layer comprises at least one compound denoted by general formula (I).

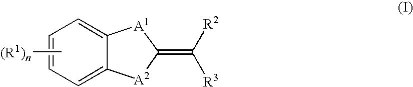

In general formula (I), each of A¹ and A² independently denotes —CR⁴R⁵—, —O—, —NR⁶—, —S—, or —C(═O)—, each of R⁴, R⁵, and R⁶ independently denotes a hydrogen atom or a substituent, R¹ denotes a substituent, n denotes an integer ranging from 0 to 4, plural substituents denoted by R¹ are identical to or different from each other when n denotes an integer of equal to or greater than 2, each of R² and R³ independently denotes a substituent having a Hammett substituent constant, σp value, of greater than 0, R² and R³ do not form a ring structure by bonding together, and at least one from among R¹, R², R³, A¹, and A² comprises at least one polymerizable group.

The above compound may have a molar absorbance coefficient of equal to or lower than 200 mole·1cm⁻¹ at a wavelength of 405 nm.

Among A¹ and A² in general formula (I), there may be the case where one denotes —S— and the other denotes —NR⁶—, or one denotes —S— and the other denotes —NR⁶—,

The above polymerizable group may be a radical polymerizable group.

The above compound may have a maximum absorption wavelength of less than 405 nm.

The above optical recording composition and/or the above recording layer may further comprise at least one photo-induced polymerization initiator.

The above photo-induced polymerization initiator may be a compound denoted by general formula (II):

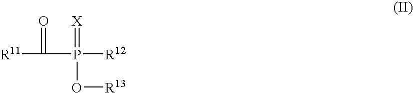

wherein, in general formula (II), each of R¹¹, R¹², and R¹³ independently denotes an alkyl group, aryl group, or heterocyclic group, and X denotes an oxygen atom or sulfur atom.

The above optical recording composition and/or the above recording layer may further comprise at least one polyfunctional isocyanate and polyfunctional alcohol.

The above optical recording composition may be a holographic recording composition.

The compound denoted by general formula (I) is capable of high-sensitivity recording when conducting holographic recording employing a recording light source in the form of a laser having a central wavelength around 405 nm, specifically, 405±20 nm. The above compound is also suited to digital volume holography, permitting the use of an inexpensive laser and a reduction in writing time.

The holographic recording medium of the present invention permits ultrahigh-density optical recording due to the presence of a holographic recording layer comprising one or more of the above compound, and is optimal as a recording medium for volume holography, particularly digital volume holography.

Other exemplary embodiments and advantages of the present invention may be ascertained by reviewing the present disclosure and the accompanying drawings.

### BRIEF DESCRIPTION OF THE DRAWINGS

The present invention will be described in the following text by the exemplary, non-limiting embodiments shown in the figures, wherein:

FIG. 1 is a schematic cross-sectional view of an example of a holographic recording medium according to a first implementation embodiment.

FIG. 2 is a schematic cross-sectional view of an example of a holographic recording medium according to a second implementation embodiment.

FIG. 3 is a drawing descriptive of an example of an optical system permitting recording and reproducing of information on a holographic recording medium.

FIG. 4 is a block diagram showing an example of the overall configuration of a recording and reproducing device suited to use in recording and reproducing information on the holographic recording medium of the present invention.

FIG. 5 is a schematic of the optical system of a planar wave tester.

Explanations of Symbols in the Drawings are as Follows:
1 Lower substrate
2 Reflective film
3 Servo pit pattern
4 Recording layer
5 Upper substrate
6 Filter layer
7 Second gap layer
8 First gap layer
12 Objective lens
13 Dichroic mirror
14 Detector
15 ¼ wavelength plate
16 Polarizing plate
17 Half mirror
20 Holographic recording medium
21 Holographic recording medium
22 Holographic recording medium
31 Pickup
81 Spindle
82 Spindle motor
83 Spindle servo circuit
84 Driving device
85 Detection circuit
86 Focus servo circuit
87 Tracking servo circuit
88 Slide servo circuit
89 Signal processing circuit
90 Controller
91 Operation element
100 Optical recording and reproducing device
A Entry and exit surface
FE Focus error signal
TE Tracking error signal
RF Reproduction signal

### DETAILED DESCRIPTIONS OF THE EMBODIMENTS

The following preferred specific embodiments are, therefore, to be construed as merely illustrative, and non-limiting to the remainder of the disclosure in any way whatsoever. In this regard, no attempt is made to show structural details of the present invention in more detail than is necessary for fundamental understanding of the present invention; the description taken with the drawings making apparent to those skilled in the art how several forms of the present invention may be embodied in practice.

### Optical Recording Composition

The optical recording composition of the present invention comprises at least one merocyanine compound denoted by general formula (I) below. The merocyanine compound denoted by general formulas (I) can be employed as a recording material in various recording systems in which information is recorded by irradiation with light. Among these, it is desirably employed as an optical recording compound such as a holographic recording compound, and is particularly suitable as a volume holographic recording compound. The holographic recording is a method of recording information by superposing an informing light containing information and a reference light in a recording layer to write an interference fringe thus formed in the recording layer. Volume holographic recording is a method of recording information in holographic recording in which a three-dimensional interference image is written in the recording layer. In the present invention, the term “holographic recording compound” refers to a compound that permits the recording of an interference fringe as refractive index modulation, either directly or indirectly, by irradiating light to record information. The compound denoted by general formula (I) can undergo a polymerization reaction, either directly or through the action of a photo-induced polymerization initiator, when irradiated with light, thereby permitting the recording of interference fringes as refractive index modulation.

The compound denoted by general formula (I) will be described in greater detail below.

### Compound Denoted by General Formula (I)

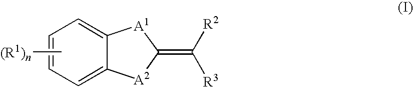

In general formula (I), each of A¹ and A² independently denotes —CR⁴R⁵—, —O—, —NR⁶—, —S—, or —C(═O)—. Each of A¹ and A² desirably denotes —CR⁴R⁵—, —O—, —NR⁶—, or —S—; preferably —O—, —NR⁶—, —S—; and more preferably, —NR⁶— or —S—.

Specific examples of combinations of A¹ and A² are (—CR⁴R⁵—, —NR⁶—), (—S—, —S—), (—S—, —NR⁶—), (—O—, NR⁶—), (—NR⁶—, —NR⁶—), (—C(═O)—, —NR⁶); desirable examples are (—S—, —S—), (—S—, —NR⁶—), (—O—, —NR⁶—), (—NR⁶—, —NR⁶—); preferred examples are (—O—, —NR⁶—), (—S—, —NR⁶—); and a further preferred example is (—S—, —NR⁶—).

In general formula (I), R¹ denotes a substituent. Each of R⁴, R⁵, and R⁶ independently denotes a hydrogen atom or a substituent.

R¹ and R⁴ to R⁶ may be selected from the group of substituents listed by way of example below:

Group of substituents: halogen atoms (such as fluorine, chlorine, bromine, and iodine atoms); alkyl groups (desirably alkyl groups having 1 to 30 carbon atoms, such as methyl, ethyl, n-propyl, isopropyl, tert-butyl, n-octyl, and 2-ethylhexyl groups); cycloalkyl groups (desirably substituted or unsubstituted cycloalkyl groups having 3 to 30 carbon atoms, such as cyclohexyl, cyclopentyl, and 4-n-dodecylcyclohexyl groups); bicycloalkyl groups (desirably substituted or unsubstituted bicycloalkyl groups having 5 to 30 carbon atoms, that is, monovalent groups obtained by removing a hydrogen atom from a bicycloalkane having 5 to 30 carbon atoms, such as bicyclo[1,2,2]heptane-2-yl and bicyclo[2,2,2]octane-3-yl groups); alkenyl groups (desirably substituted or unsubstituted alkenyl groups having 2 to 30 carbon atoms, such as vinyl and allyl groups); cycloalkenyl groups (desirably substituted or unsubstituted cycloalkenyl groups having 3 to 30 carbon atoms, that is, monovalent groups obtained by removing a hydrogen atom from a cycloalkene having 3 to 30 carbon atoms, such as 2-cycloheptene-1-yl and 2-cyclohexene-1-yl groups); bicycloalkenyl groups (substituted or unsubstituted bicycloalkenyl groups, desirably substituted or unsubstituted bicycloalkenyl groups having 5 to 30 carbon atoms, that is, monovalent groups obtained by removing a hydrogen atom from a bicycloalkene having a single double bond, such as bicyclo[2,2,1]hepto-2-ene-1-yl and bicyclo[2,2,2]octo-2-ene-4-yl groups); alkynyl groups (desirably substituted or unsubstituted alkynyl groups having 2 to 30 carbon atoms, such as ethynyl and propargyl groups); aryl groups (desirably substituted or unsubstituted aryl groups having 6 to 30 carbon atoms, such as phenyl, p-tolyl, and naphthyl groups); heterocyclic groups (desirably five or six-membered, substituted or unsubstituted monovalent groups obtained by removing a hydrogen atom from an aromatic or nonaromatic heterocyclic compound; preferably five or six-membered aromatic heterocyclic groups having 3 to 30 carbon atoms; such as 2-furyl, 2-thienyl, 2-pyridinyl, and 2-benzothazolyl groups); cyano groups; hydroxyl groups; nitro groups; carboxyl groups; alkoxy groups (desirably substituted or unsubstituted alkoxy groups having 1 to 30 carbon atoms, such as methoxy, ethoxy, isopropoxy, tert-butoxy, n-octyloxy, and 2-methoxyethoxy groups); aryloxy groups (desirably substituted or unsubstituted aryloxy groups having 6 to 30 carbon atoms, such as phenoxy, 2-methylphenoxy, 4-tert-butylphenoxy, 3-nitrophenoxy, and 2-tetradecanoylaminophenoxy groups); silyloxy groups (desirably silyloxy groups having 3 to 20 carbon atoms, such as trimethylsilyloxy and tert-butyldimethylsilyloxy groups); heterocyclic oxy groups (desirably substituted or unsubstituted heterocyclic oxy groups having 2 to 30 carbon atoms, such as 1-phenyltetrazole-5-oxy and 2-tetrahydropyranyloxy groups); acyloxy groups (desirably formyloxy groups, substituted or unsubstituted alkylcarbonyloxy groups having 2 to 30 carbon atoms, and substituted or unsubstituted arylcarbonyloxy groups having 6 to 30 carbon atoms, such as formyloxy, acetyloxy, pivaloyloxy, stearoyloxy, benzoyloxy, and p-methoxyphenylcarbonyloxy groups); carbamoyloxy groups (desirably substituted or unsubstituted carbamoyloxy groups having 1 to 30 carbon atoms, such as N,N-dimethylcarbamoyloxy, N,N-diethylcarbamoyloxy, morpholinocarbonyloxy, N,N-di-n-octylaminocarbonyloxy, and N-n-octylcarbamoyloxy groups); alkoxycarbonyloxy groups (desirably substituted or unsubstituted alkoxycarbonyloxy groups having 2 to 30 carbon atoms, such as methoxycarbonyloxy, ethoxycarbonyloxy, tert-butoxycarbonyloxy, and n-octylcarbonyloxy groups), aryloxycarbonyloxy groups (desirably substituted or unsubstituted aryloxycarbonyloxy groups having 7 to 30 carbon atoms, such as phenoxycarbonyloxy, p-methoxyphenoxycarbonyloxy, and p-n-hexadecyloxyphenoxycarbonyloxy groups); amino groups (desirably amino groups, substituted or unsubstituted alkylamino groups having 1 to 30 carbon atoms, and substituted or unsubstituted anilino groups having 6 to 30 carbon atoms, such as amino, methylamino, dimethylamino, anilino, N-methylanilino, and diphenylamino groups); acylamino groups (desirably formylamino groups, substituted or unsubstituted alkylcarbonylamino groups having 1 to 30 carbon atoms, and substituted or unsubstituted arylcarbonylamino groups having 6 to 30 carbon atoms, such as formylamino, acetylamino, pivaloylamino, lauroylamino, and benzoylamino groups); aminocarbonylamino groups (desirably substituted or unsubstituted aminocarbonylamino groups having 1 to 30 carbon atoms, such as carbamoylamino, N,N-dimethylaminocarbonylamino, N,N-diethylaminocarbonylamino, and morpholinocarbonylamino groups); alkoxycarbonylamino groups (desirably substituted or unsubstituted alkoxycarbonylamino groups having 2 to 30 carbon atoms, such as methoxycarbonylamino, ethoxycarbonylamino, tert-butoxycarbonylamino, n-octadecyloxycarbonylamino, and N-methylmethoxycarbonylamino groups); aryloxycarbonylamino groups (desirably substituted or unsubstituted aryloxycarbonyl-amino groups having 7 to 30 carbon atoms, such as phenoxycarbonylamino, p-chloro-phenoxycarbonylamino, and m-n-octyloxyphenoxycarbonylamino groups); sulfamoylamino groups (desirably substituted or unsubstituted sulfamoylamino groups having 0 to 30 carbon atoms, such as sulfamoylamino, N,N-dimethylaminosulfonylamino, and N-n-octylaminosulfonylamino groups); alkyl and arylsulfonylamino groups (desirably substituted or unsubstituted alkylsulfonylamino groups having 1 to 30 carbon atoms and substituted or unsubstituted arylsulfonylamino groups having 6 to 30 carbon atoms, such as methylsulfonylamino, butylsulfonylamino, phenylsulfonylamino, 2,3,5-trichlorophenylsulfonylamino, and p-methylphenylsulfonylamino groups); merearpto groups; alkylthio groups (desirably substituted or unsubstituted alkylthio groups having 1 to 30 carbon atoms, such as methylthio, ethylthio, and n-hexadecylthio groups); arylthio groups (desirably substituted or unsubstituted arylthio groups having 6 to 30 carbon atoms, such as phenylthio, p-chlorophenylthio, and m-methoxyphenylthio groups); heterocyclic thio groups (desirably substituted or unsubstituted heterocyclic thio groups having 2 to 30 carbon atoms, such as 2-benzothiazolylthio and 1-phenyltetrazol-5-ylthio groups); sulfamoyl groups (desirably substituted or unsubstituted sulfamoyl groups having 0 to 30 carbon atoms, such as N-ethylsulfamoyl, N-(3-dodecyloxypropyl)sulfamoyl, N,N-dimethylsulfamoyl, N-acetylsulfamoyl, N-benzoylsulfamoyl, and N-(N′-phenylcarbamoyl)sulfamoyl groups); sulfo groups; alkyl and arylsulfinyl groups (desirably substituted or unsubstituted alkylarylsulfinyl groups having 1 to 30 carbon atoms and substituted or unsubstituted arylsulfinyl groups having 6 to 30 carbon atoms, such as methylsulfinyl, ethylsulfinyl, phenylsulfinyl, and p-methylphenylsulfinyl groups); alkyl and arylsulfonyl groups (desirably substituted or unsubstituted alkylsulfonyl groups having 1 to 30 carbon atoms and substituted or unsubstituted arylsulfonyl groups having 6 to 30 carbon atoms, such as methylsulfonyl, ethylsulfonyl, phenylsulfonyl, and p-methylphenylsulfonyl groups); acyl groups (desirably formyl group, substituted or unsubstituted alkylcarbonyl groups having 2 to 30 carbon atoms and substituted or unsubstituted arylcarbonyl groups having 7 to 30 carbon atoms, such as acetyl and pivaloylbenzoyl groups); aryloxycarbonyl groups (desirably aryloxycarbonyl groups having 7 to 30 carbon atoms, such as phenoxycarbonyl, o-chlorophenoxycarbonyl, m-nitrophenoxycarbonyl, and p-tert-butylphenoxycarbonyl groups); alkoxycarbonyl groups (desirably substituted or unsubstituted alkoxycarbonyl groups having 2 to 30 carbon atoms, such as methoxycarbonyl, ethoxycarbonyl, tert-butoxycarbonyl, and n-octadecyloxycarbonyl groups); carbamoyl groups (desirably substituted or unsubstituted carbamoyl groups having 1 to 30 carbon atoms, such as carbamoyl, N-methylcarbamoyl, N,N-dimethylcarbamoyl, N,N-di-n-octylcarbamoyl, and N-(methylsulfonyl)carbamoyl groups); aryl and heterocyclic azo groups (desirably substituted or unsubstituted arylazo groups having 6 to 30 carbon atoms and substituted or unsubstituted heterocyclic azo groups having 3 to 30 carbon atoms, such as phenylazo, p-chlorophenylazo, and 5-ethylthio-1,3,4-thiadiazol-2-ylazo groups); imide groups (desirably N-succinimide and N-phthalimide groups); phosphino groups (desirably substituted or unsubstituted phosphino groups having 2 to 30 carbon atoms, such as dimethylphosphino, diphenylphosphino, and methylphenoxyphosphino groups); phosphinyl groups (desirably substituted or unsubstituted phosphinyl groups having 2 to 30 carbon atoms, such as phosphinyl, dioctyloxyphosphinyl, and diethoxyphosphinyl groups); phosphinyloxy groups (desirably substituted or unsubstituted phosphinyloxy groups having 2 to 30 carbon atoms, such as diphenoxyphosphinyloxy and dioctyloxyphosphinyloxy groups); phosphinylamino groups (desirably substituted or unsubstituted phosphinylamino groups having 2 to 30 carbon atoms, such as dimethoxyphosphinylamino and dimethylaminophosphinylamino groups); and silyl groups (desirably substituted or unsubstituted silyl groups having 3 to 30 carbon atoms, such as trimethylsilyl, tert-butyldimethylsilyl, and phenyldimethylsilyl groups).

In those of the above-listed substituents having a hydrogen atom, the hydrogen atom may be removed and one of the above-listed substituents substituted in its place. Examples of such functional groups are: alkylcarbonylaminosulfonyl, arylcarbonyl-aminosulfonyl, alkylsulfonylaminocarbonyl, and arylsulfonylaminocarbonyl groups. Examples thereof are: methylsulfonylaminocarbonyl, p-methylphenylsulfonyl-aminocarbonyl, acetylaminosulfonyl, and benzoylaminosulfonyl groups. In the present invention, the number of carbon atoms indicated for a given group means the number of carbon atoms of the portion of that group excluding substituents.

In general formula (I), n denotes an integer ranging from 0 to 4; desirably, an integer ranging from 0 to 2; and preferably, 0 or 1. When n denotes an integer of equal to or greater than 2, plural substituents denoted by R¹ may be identical to or different from each other.

R¹ desirably denotes a halogen atom, alkyl group, alkenyl group, aryl group, heterocyclic group, hydroxyl group, carboxyl group, acyl group, alkoxy group, aryloxy group, acyloxy group, cyano group, or amino group; preferably denotes a halogen atom, alkyl group, cyano group, alkoxy group, or acyloxy group; and more preferably, denotes a halogen atom, alkoxy group, or acyloxy group.

Each of R⁴ to R⁶ desirably denotes an alkyl group, cycloalkyl group, bicycloalkyl group, alkenyl group, cycloalkenyl group, bicycloalkenyl group, alkynyl group, aryl group, or heterocyclic group. R⁴ to R⁶ preferably denote alkyl groups.

In general formula (I), each of R² and R³ independently denotes a substituent having a Hammett substituent constant, op value, of greater than 0. The Hammett σp value is described in detail in, for example, works such as N. Inamoto, “Hammett's Rule—Structure and Reactivity—,” (Maruzen); Chemical Society of Japan, comp., “New Experimental Chemistry Lecture 14, Synthesis and Reaction of Organic Compounds V,” p. 2605 (Maruzen); T. Nakatani, “An Expository of Theoretical Organic Chemistry,” p. 217 (Tokyo Kagaku Dojin); and Chemical Revue, Vol. 91, 165-195, (1991), which are expressly incorporated herein by reference in their entirety. When R² and R³ both denote electron-withdrawing substituents having Hammett op values of greater than 0, absorption by the compound at the wavelength around 400 nm decreases, making it possible to achieve a medium with high transmittance in the short wavelength region. The above substituents preferably have an electron-withdrawing property in the form of a σp value of greater than 0 and equal to or less than 1.5. More preferably, they are selected from among the substituents indicated further below. Still more preferably, they are acyl, oxycarbonyl, carbamoyl, cyano, or sulfonyl groups. However, in general formula (I), R² and R³ do not form a ring structure by bonding together. The compound denoted by general formula (I) can be employed with particular preference in recording and reproduction employing light with a wavelength of around 400 nm. However, when R² and R³ bond together, the absorption of the compound tends to increase in wavelength and absorb light with a wavelength of around 400 nm, compromising absorption efficiency.

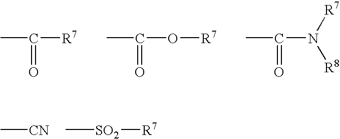

Each of R⁷ and R⁸ above independently denotes a hydrogen atom or a substituent; desirably denotes a hydrogen atom, alkyl group having 1 to 30 carbon atoms, substituted or unsubstituted cycloalkyl group having 3 to 30 carbon atoms, substituted or unsubstituted cycloalkyl group having 3 to 30 carbon atoms, substituted or unsubstituted alkenyl group having 2 to 30 carbon atoms, substituted or unsubstituted aryl group having 6 to 30 carbon atoms, or five or six-membered, substituted or unsubstituted, aromatic or nonaromatic heterocyclic group; preferably denotes an alkyl group having 1 to 20 carbon atoms, substituted or unsubstituted cycloalkyl group having 3 to 20 carbon atoms, or substituted or unsubstituted alkenyl group having 2 to 20 carbon atoms; and more preferably, denotes an alkyl group having 1 to 15 carbon atoms, substituted or unsubstituted cycloalkyl group having 3 to 15 carbon atoms, or substituted or unsubstituted alkenyl group having 2 to 15 carbon atoms.

The combination of R² and R³ is desirably selected from among: (cyano group, cyano group), (cyano group, oxycarbonyl group), (cyano group, acyl group), (cyano group, carbamoyl group), (cyano group, sulfonyl group), (oxycarbonyl group, oxycarbonyl group), (oxycarbonyl group, acyl group), (oxycarbonyl group, sulfonyl group), (carbamoyl group, carbamoyl group), (carbamoyl group, acyl group), and (acyl group, acyl group); preferably selected from among: (cyano group, cyano group), (cyano group, oxycarbonyl group), (cyano group, carbamoyl group), (oxycarbonyl group, oxycarbonyl group), (oxycarbonyl group, acyl group), (oxycarbonyl group, sulfonyl group), (carbamoyl group, carbamoyl group), and (acyl group, acyl group); more preferably selected from among: (cyano group, oxycarbonyl group), (oxycarbonyl group, oxycarbonyl group), and (oxycarbonyl group, sulfonyl group); and still more preferably, is a (cyano group, oxycarbonyl group).

In general formula (I), at least one from among R¹, R², R³, A¹, and A² comprises at least one polymerizable group. When each of A¹ and A² independently denotes —CR⁴R⁵— or —NR⁶—, R⁴, R⁵, and/or R⁶ may contain a polymerizable group. In the present invention, the term “polymerizable group” means a substituent capable of imparting a polymerization property to the compound denoted by general formula (I) so that it can be polymerized. More specifically, it is a substituent capable of causing a polymerizable component to be polymerized by irradiation with light, irradiation with radiation, heating, the use of a radical initiator, or the like. From the perspective of not undergoing reaction in the dark, a radical polymerizable group is desirable. A functional group capable of undergoing addition polymerization or condensation polymerization is preferred. The presence of a polymerizable group permits the direct or indirect recording of an interference fringe as refractive index modulation by irradiation with a recording light.

The above polymerizable group is desirably a functional group capable of undergoing addition polymerization. A polymerizable ethylenic unsaturated group or ring-opening polymerizable group is preferred as the polymerizable group, with a polymerizable ethylenic unsaturated group being of greater preference. Specifically, an acrylic group, methacrylic group, or styryl group is desirable; an acrylic group or methacrylic group is preferable; and an acrylic group is of greater preference. The substitution position of the polymerizable group is not specifically limited. In the compound denoted by general formula (I), one or more from among R² to R⁶ desirably comprises at least one polymerizable group, and R², R³ or R⁶ preferably comprises at least one polymerizable group. Examples of the polymerizable group are given below.

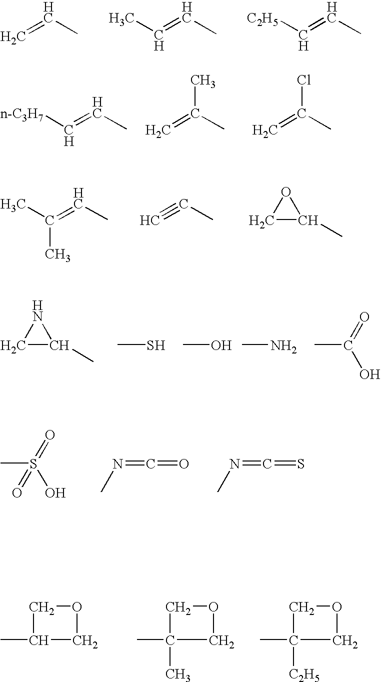

Formulas (M-1) to (M-6) below are examples of polymerizable ethylenic unsaturated groups.

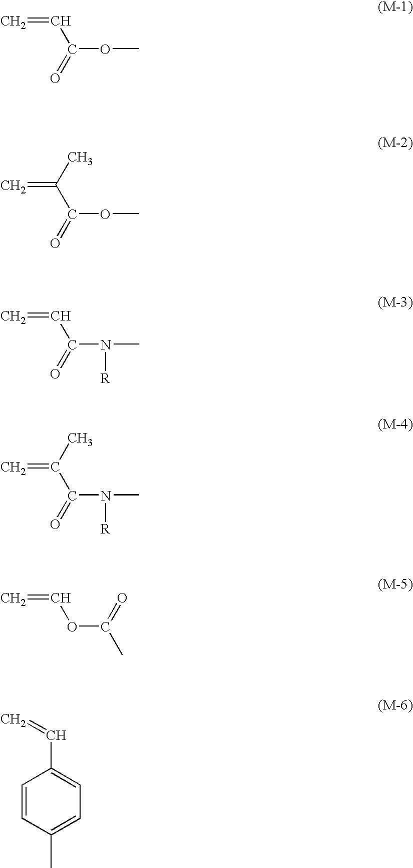

In formulas (M-3) to (M-4), R denotes a hydrogen atom or an alkyl group, desirably a hydrogen atom or a methyl group. Among formulas (M-1) to (M-6), an acrylic group denoted by formula (M-1), a methacrylic group denoted by formula (M-2), or a styryl group denoted by formula (M-6) is desirable, with an acrylic group denoted by formula (M-1) or a methacrylic group denoted by formula (M-2) being preferred Specific examples of the compound denoted by general formula (I) are given below. However, the present invention is not limited to these examples.

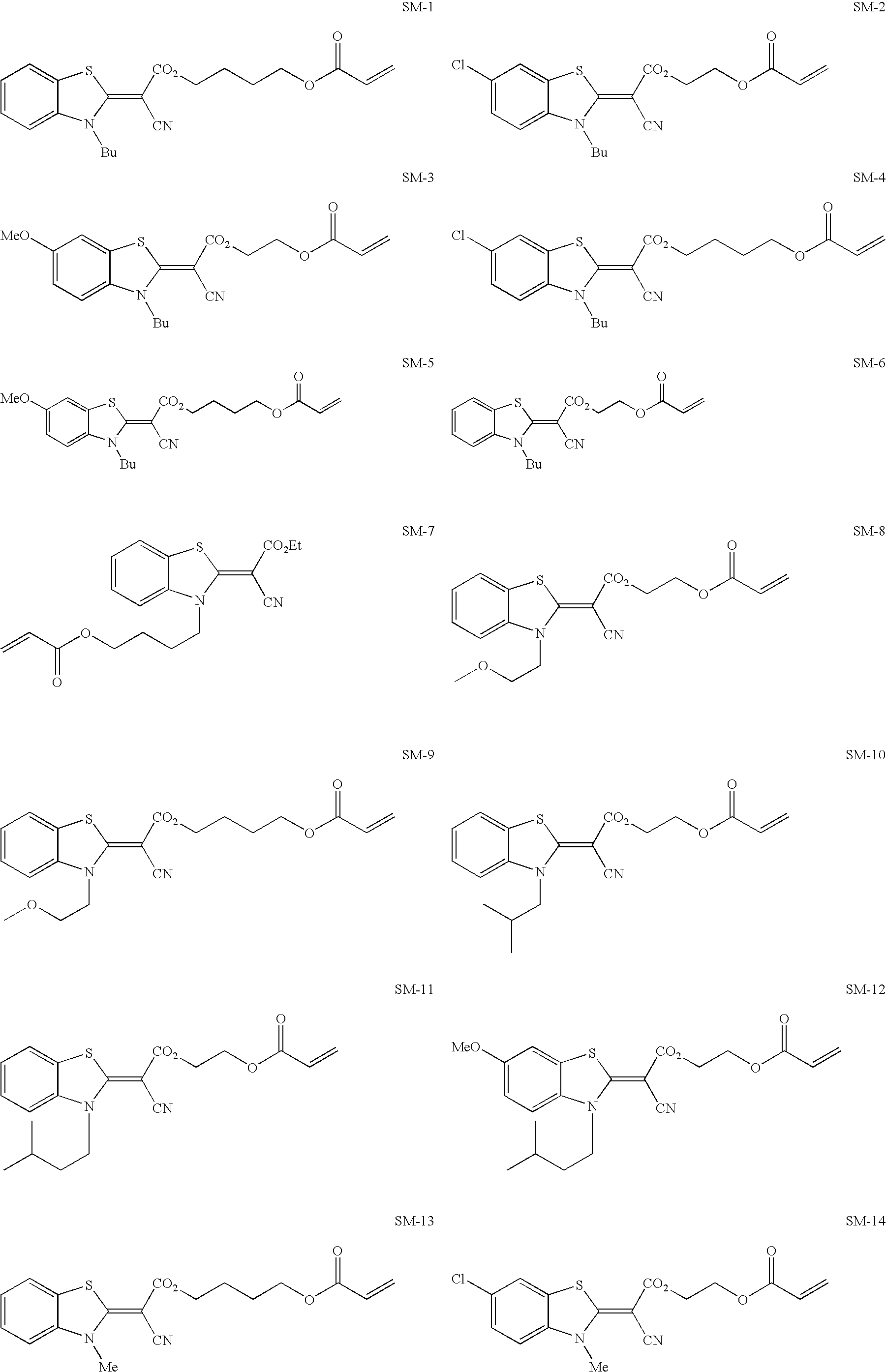

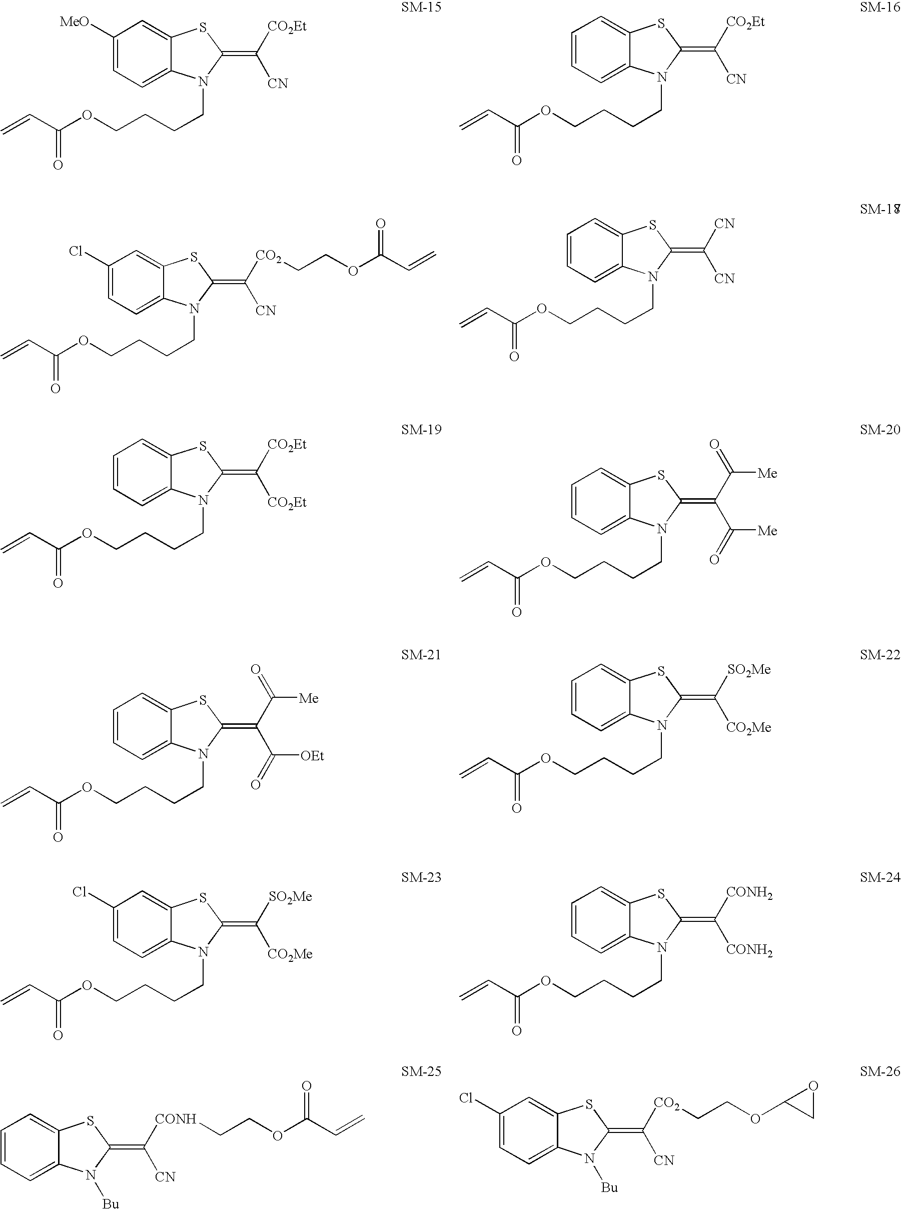

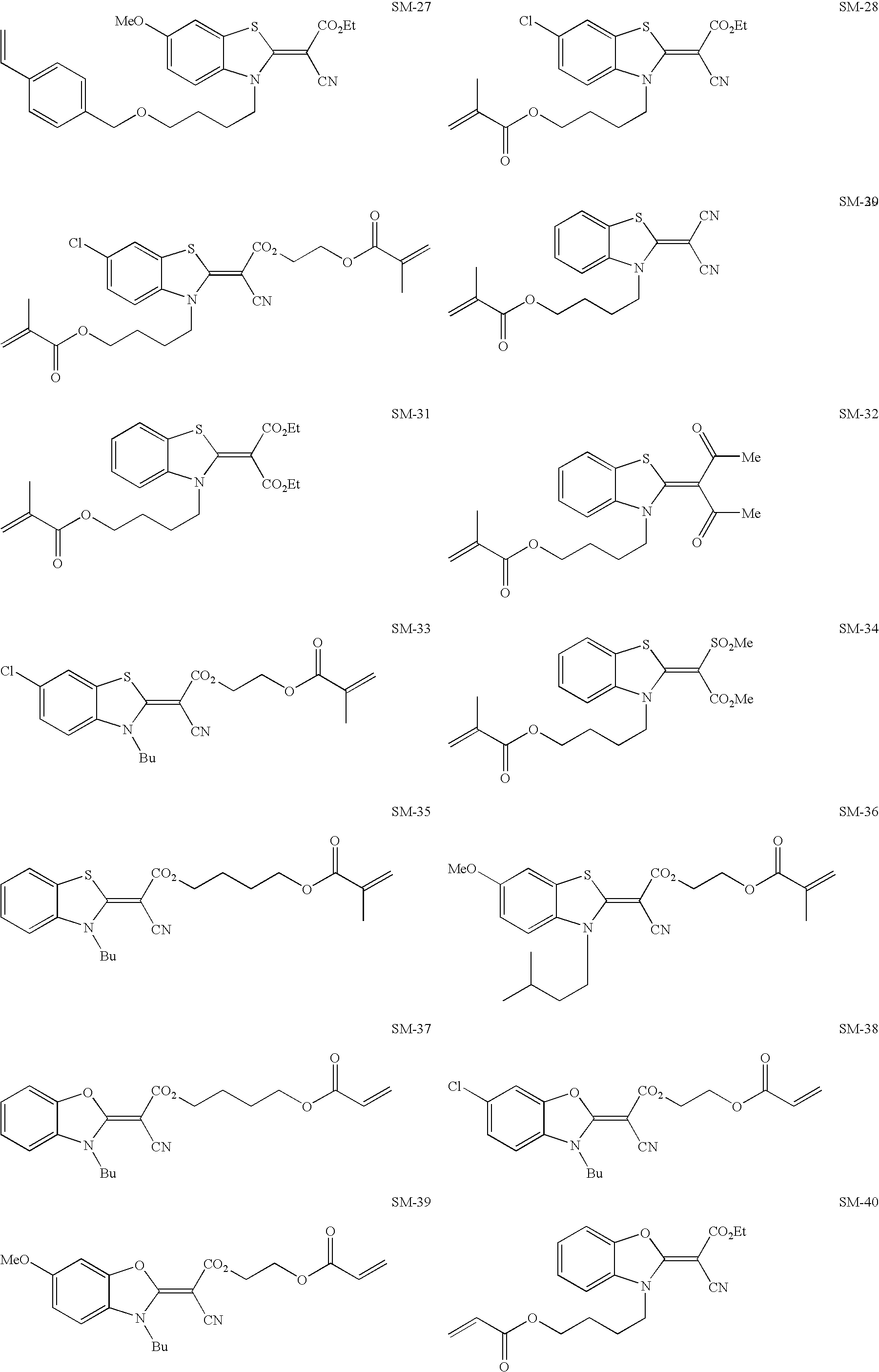

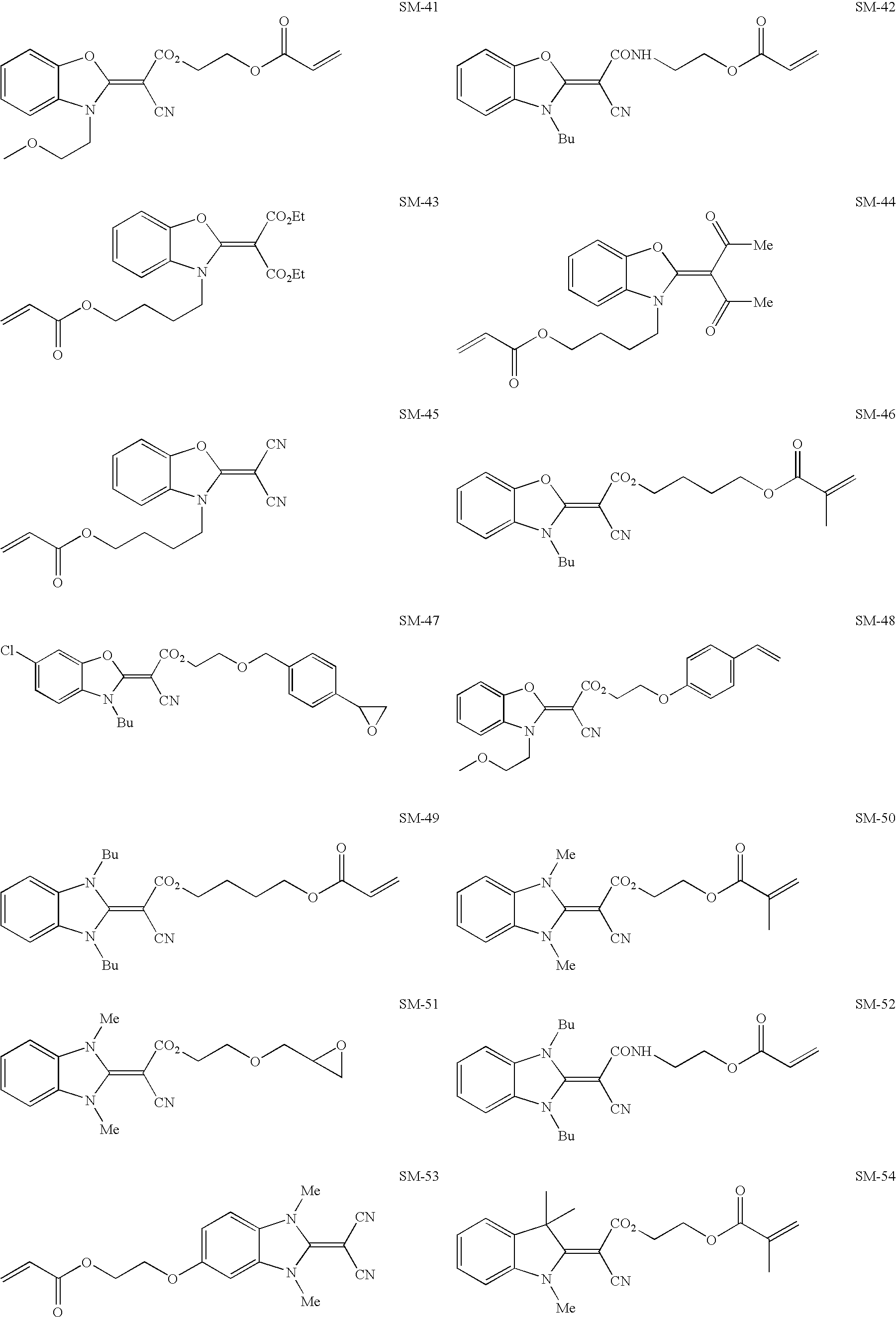

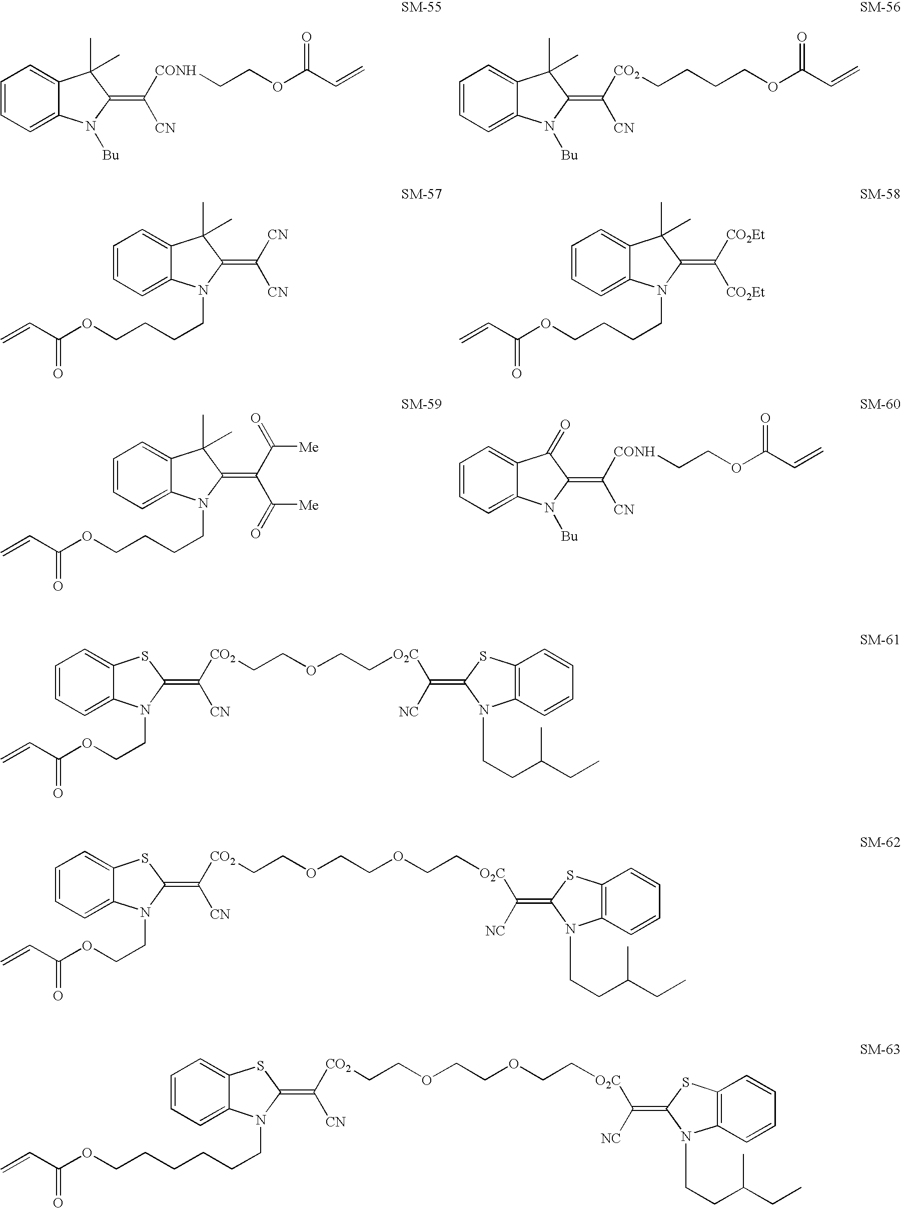

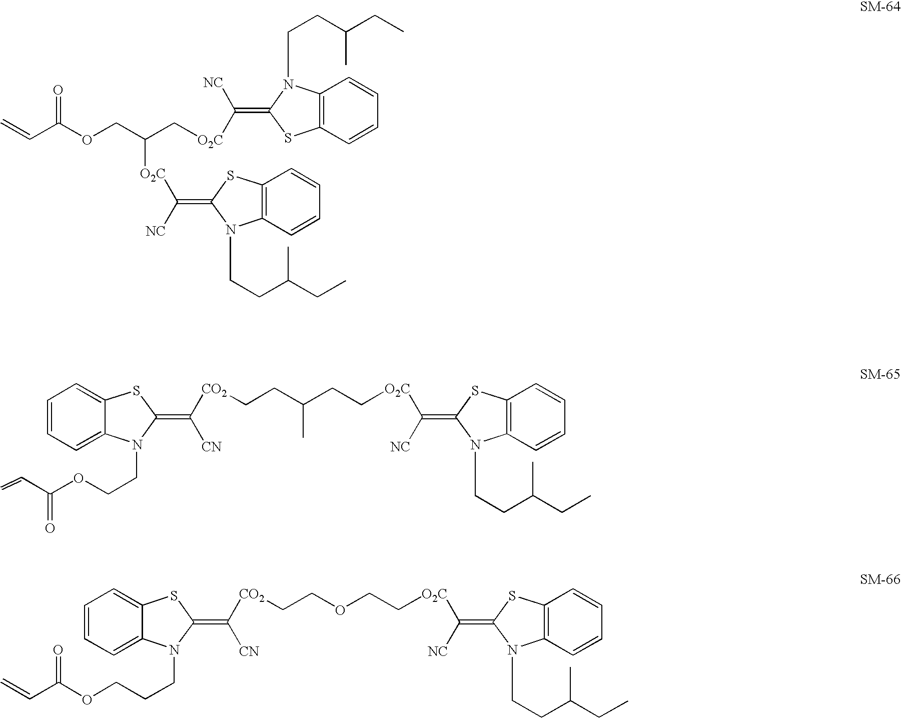

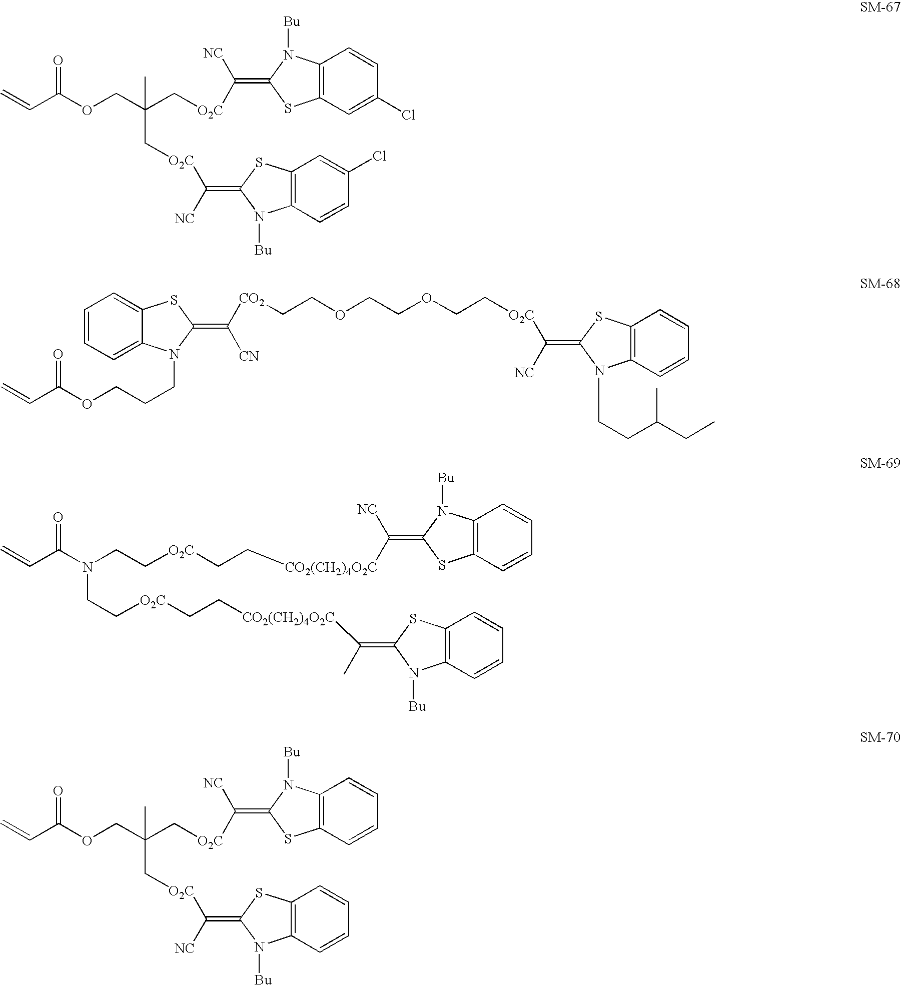

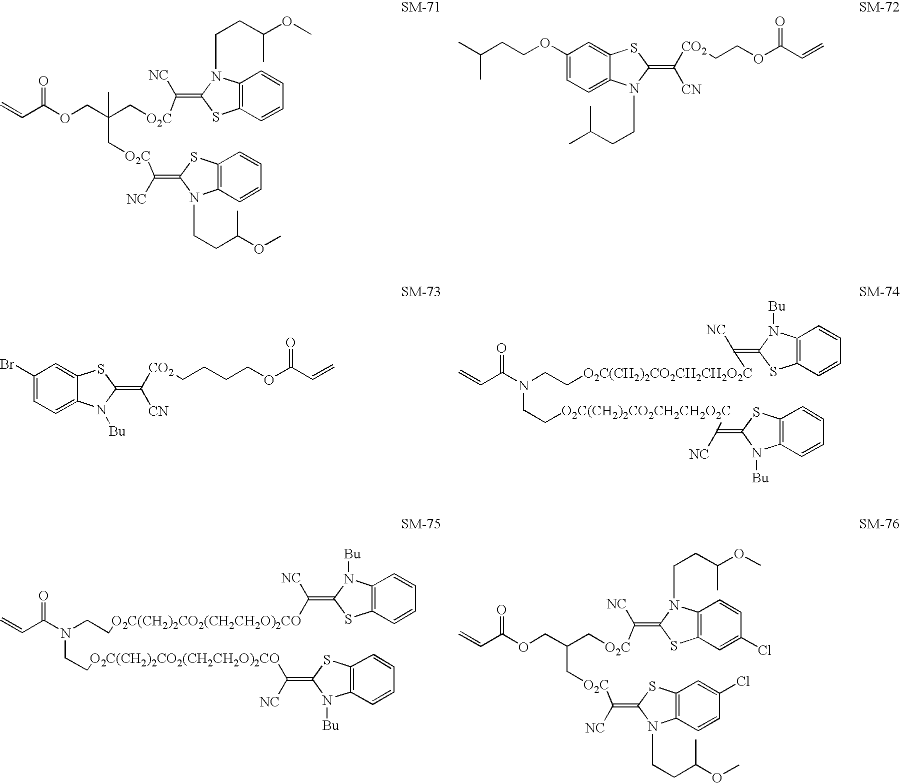

The compound denoted by general formula (I) can be synthesized by a combination of various known methods. The optimal synthesis method can be selected based on the individual compound. Schemes 1 and 2 below are examples of synthesis methods.

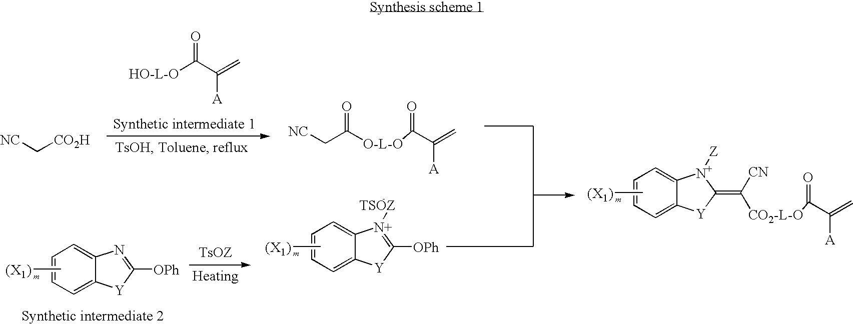

In synthesis scheme 1 above, L denotes a divalent linking group (such as a group comprised of a combination of groups selected from among alkylene, arylene, —O—, —S—, —C(═O)—, —SO₂—, and —NH— (where an alkyl group or the like can be substituted for the hydrogen atom in —NH—)); A denotes a hydrogen atom or an alkyl group; X₁ denotes a substituent; m denotes an integer ranging from 0 to 4; and Y denotes a sulfur atom, oxygen atom, NQ group, CQQ′ group, or C═O group. Each of Q and Q′ independently denotes an alkyl group, alkenyl group, alkynyl group, aryl group, or heterocyclic group. Z denotes an alkyl group. Synthetic intermediate 1 can be synthesized by selecting suitable starting materials by referring to Acta Chim. Hung. 1986, No. 122, p. 65, which is expressly incorporated herein by reference in its entirety. Synthetic intermediate 2 can be synthesized by selecting suitable starting materials by referring to J. Am. Chem. Soc. 1949, No. 71, p. 3340; Synthesis 1994, p. 1124; J. Hetercycl. Chem. 1987, No. 24, p. 275, which are expressly incorporated herein by reference in their entirety, or the like.

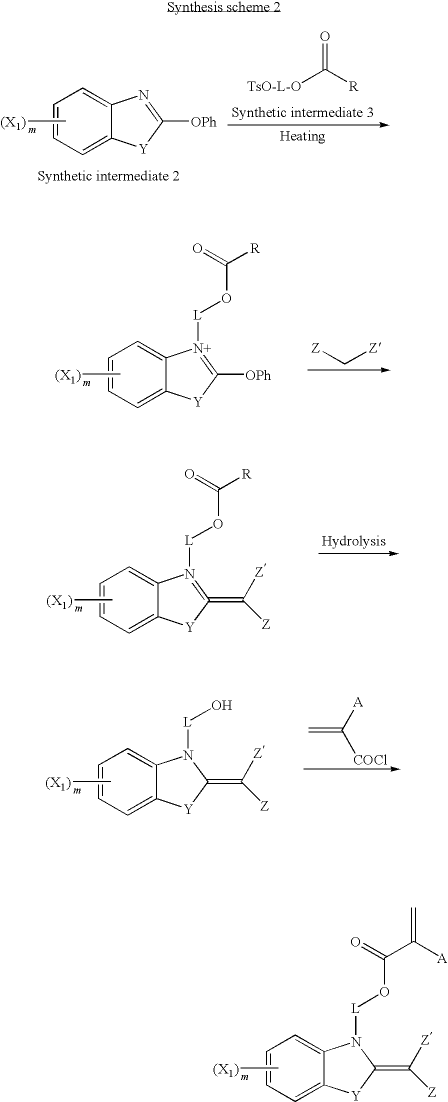

In synthesis scheme 2 above, L denotes a divalent linking group (such as a group comprised of a combination of groups selected from among alkylene, arylene, —O—, —S—, —C(═O)—, —SO₂—, and —NH— (where an alkyl group or the like can be substituted for the hydrogen atom in —NH—)); A denotes a hydrogen atom or an alkyl group; X₁ denotes a substituent; m denotes an integer ranging from 0 to 4; and Y denotes a sulfur atom, oxygen atom, NQ group, CQQ′ group, or C═O group. Each of Q and Q′ independently denotes an alkyl group, alkenyl group, alkynyl group, aryl group, or heterocyclic group. Each of Z and Z′ independently denotes an electron-withdrawing group, more specifically, can be selected from among cyano, oxycarbonyl, carbamoyl, sulfonyl, and acyl groups. Synthetic intermediate 3 can be synthesized by selecting suitable starting materials by referring to the J. Am. Chem. Soc. 1968, No. 90, p. 3878, which is expressly incorporated herein by reference in its entirety.

Absorption at the recording wavelength in the compound employed as the recording compound in a holographic recording medium is desirably low so as to increase medium transmittance and achieve high sensitivity. The compound denoted by general formula (I) above can exhibit a molar absorbance coefficient ε of equal to or lower than 200 mole·1·cm⁻¹ at a wavelength of 405 nm, for example, and is thus suited to recording at a wavelength of around 400 nm. To achieve high recording capacity, it is desirable for the recording compound to have a great absorption at a wavelength shorter than the recording wavelength. The compound denoted by general formula (I) above can have a maximum absorption wavelength λ max of less than 405 nm, which is suitable for recording at a wavelength of around 400 nm. Specifically, the molar absorbance coefficient ε_{at 405 nm} at a wavelength of 405 nm of the compound denoted by general formula (I) is desirably equal to or lower than 200 mole·1·cm⁻¹, preferably falling within a range of 0to 100 mole·1·cm⁻¹. Further, the compound denoted by general formula (I) desirably has a maximum absorption wavelength λ max of less than 405 nm, preferably falling within a range of 300 to 350 nm. The molar absorbance coefficient at λ max is desirably equal to or greater than 10,000 mole·1·cm⁻¹, preferably equal to or greater than 30,000 mole·1·cm⁻¹. The upper limit of the molar absorbance coefficient at λ max is not specifically limited. By way of example, it is about 200,000 mole·1·cm⁻¹.

The above absorption characteristics can be obtained from absorption spectra measured with a UV-visible light spectrophotometer for a solution obtained by dissolving the compound in a suitable solvent, such as methylene chloride.

The optical recording composition of the present invention comprises at least one compound denoted by general formula (I). Just one of the compound denoted by general formula (I) can be employed, or two or more such compounds can be employed in combination. The content of the compound denoted by general formula (I) in the optical recording composition of the present invention is not specifically limited and may be suitably selected based on the objective. A content of 1 to 50 weight percent is desirable, 1 to 30 weight percent is preferable, and 2 to 10 weight percent is of even greater preference. A content of equal to or less than 50 weight percent can readily ensure a stable interference image, and a content of equal to or greater than 1 weight percent can yield desirable properties from the perspective of diffraction efficiency.

### Photo-Induced Polymerization Initiator

The optical recording composition of the present invention is desirably employed as a holographic recording composition, and is particularly suited to use as a volume holographic recording composition. When employing the optical recording composition of the present invention as a holographic recording composition, the compound denoted by general formula (I) can function as recording monomers. The optical recording composition of the present invention can comprise at least one photo-induced polymerization initiator in addition to the compound denoted by general formula (I). The photo-induced polymerization initiator is not specifically limited, beyond that it be sensitive to the recording light. A material inducing a radical polymerization reaction, cationic ring-opening polymerization reaction, or the like when radiated with light can be employed. A photo-induced radical polymerization initiator is desirable from the perspective of polymerization reaction efficiency.

Examples of such photo-induced radical polymerization initiators are: 2,2′-bis(o-chlorophenyl)-4,4′-5,5′-tetraphenyl-1,1′-biimidazole, 2,4,6-tris(trichloromethyl)-1,3,5-triazine, 2,4-bis(trichloro-methyl)-6-(p-methoxyphenylvinyl)-1,3,5-triazine, diphenyliodoniumtetrafluoroborate, diphenyliodoniumhexafluorophosphate, 4,4′-di-t-butyldiphenyliodoniumtetrafluoroborate, 4-diethylarninophenylbenzenediazoniumhexafluorophosphate, benzoin, 2-hydroxy-2-methyl-1-phenylpropane-2-one, benzophenone, thioxanthone, 2,4,6-trimethylbenzoyl diphenylacyl phosphine oxide, triphenylbutylborate tetraethyl ammonium, diphenyl-4-phenylthiophenyl sulfonium hexafluorophosphate, 2,2-dimethoxy-1,2-diphenylethane-1-one, phenylglyoxylic acid methyl ester, 2-methyl-1-[4-(methylthio)phenyl]-2-morpholinopropane-1-one, bis(2,4,6-trimethylbenzoyl)phenylphosphine oxide, 2,4,6-trimethylbenzoyl diphenyl phosphine oxide, 1,2-octanedione, 1-[4-(phenylthio)-2-(0-benzoyloxime)], and bis(η5-2,4-cyclopentadien-1-yl)bis[2,6-difluoro-3-(1H-pyrrol-1-yl)phenyltitanium]. These may be employed singly or in combinations of two or more. A sensitizing dye, described further below, may also be employed in combination based on the wavelength of the light being irradiated.

Among photo-induced radical polymerization initiators, the suitable photo-induced radical polymerization initiator may be a compound denoted by general formula (II).

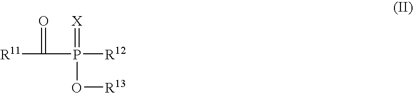

In general formula (II), each of R¹¹, R¹² and R¹³ independently denotes an alkyl group, aryl group or heterocyclic group, and X denotes an oxygen atom or sulfur atom.

The compound denoted by general formula (II) will be described in detail below.

In general formula (II), each of R²¹¹, R²¹², and R²¹³ independently denotes an alkyl group, aryl group, or heterocyclic group.

The alkyl groups denoted by R¹¹, R¹², and R¹³ can be linear or branched, and substituted or unsubstituted. They desirably have 1 to 30 carbon atoms, preferably 1 to 20 carbon atoms.

Examples of the alkyl groups denoted by R¹¹, R¹², and R¹³ are: methyl groups, ethyl groups, normal propyl groups, isopropyl groups, normal butyl groups, isobutyl groups, tertiary butyl groups, pentyl groups, cyclopentyl groups, hexyl groups, cyclohexyl groups, heptyl groups, octyl groups, tertiary octyl groups, 2-ethylhexyl groups, decyl groups, dodecyl groups, octadecyl groups, 2,3-dibromopropyl groups, adamantyl groups, benzyl groups, and 4-bromobenzyl groups. These may be further substituted. Of these, tertiary butyl groups are greatly preferred from the perspective of stability in the presence of nucleophilic compounds, such as water and alcohol.

The aryl groups denoted by R¹¹, R¹², and R¹³ in general formula (II) can be substituted or unsubstituted. They desirably comprise 6 to 30 carbon atoms, preferably 6 to 20 carbon atoms. Specific examples of these aryl groups are: phenyl groups, naphthyl groups, and anthranyl groups. These may be further substituted. Of these, R¹¹ desirably denotes an aryl group in which an alkyl group, aryl group, alkoxy group, or halogen group is present at position 2, and preferably denotes an aryl group in which an alkyl group, aryl group, alkoxy group, or halogen group is present at positions 2 and 6. For example, R¹¹ desirably denotes a 2-methylphenyl group, 2,4,6-trimethylphenyl group, 2,6-dichlorophenyl group, 2,6-dimethoxyphenyl group, or 2,6-trifluoromethylphenyl group, and preferably denotes a 2,4,6-trimethylphenyl group, 2,6-dichlorophenyl group, or 2,6-dimethoxyphenyl group. The presence of the above substituents at position 2, or at positions 2 and 6, is desirable to enhance stability in the presence of nucleophilic compounds, such as water and alcohols, as described in, for example, Jacobi, M., Henne, A. Polymers Paint Colour Journal 1985, 175, 636, which is expressly incorporated herein by reference in its entirety. Details of desirable examples of alkyl groups and aryl groups employed as the above substituents are identical to those set forth for the alkyl groups and aryl groups denoted by R¹¹, R¹², and R¹³ above.

The heterocyclic groups denoted by R¹¹, R¹², and R¹³ in general formula (II) are desirably four to eight-membered rings, preferably four to six-membered rings, and more preferably, five or six-membered rings. Specific examples are: pyridine rings, piperazine rings, thiophene rings, pyrrole rings, imidazole rings, oxazole rings, and thiazole rings. They may be further substituted. Of these hetero rings, pyridine rings are particularly desirable.

When the groups denoted by R¹¹, R¹², and R¹³ in general formula (II) comprise one or more substituents, examples of the substituents are: halogen groups, alkyl groups, alkenyl groups, alkoxy groups, aryloxy groups, alkylthio groups, alkoxycarbonyl groups, aryloxycarbonyl groups, amino groups, acyl groups, alkylaminocarbonyl groups, arylaminocarbonyl groups, sulfonamide groups, cyano groups, carboxy groups, hydroxyl groups, and sulfonic acid groups. Of these, halogen groups, alkoxy groups, and alkylthio groups are particularly desirable. When R¹¹ denotes an aryl group as set forth above, the above substituents are desirably present at position 2, or positions 2 and 6, on the aryl group.

In general formula (II), X denotes an oxygen atom or a sulfur atom, desirably an oxygen atom.

Examples of desirable compounds denoted by general formula (II) are compounds in which R¹¹ denotes an aryl group with an alkyl group, aryl group, alkoxy group, or halogen group present at position 2, R¹² denotes an aryl group, R¹³ denotes an alkyl group, and X denotes an oxygen atom or a sulfur atom. Examples of preferred compounds are compounds in which R¹¹ denotes an aryl group with an alkyl group, aryl group, alkoxy group, or halogen group present at positions 2 and 6, R¹² denotes an aryl group, R¹³ denotes an alkyl group, and X denotes an oxygen atom. Examples of compounds of greater preference are compounds in which R¹¹ denotes a 2,6-dimethoxybenzoyl group or 2,6-dichlorobenzoyl group, R¹² denotes a phenyl group, R¹³ denotes an ethyl group or isopropyl group, and X[ denotes an oxygen atom.

Specific examples of the phosphorus compound denoted by general formula (II) are given below. However, the present invention is not limited to these specific examples.

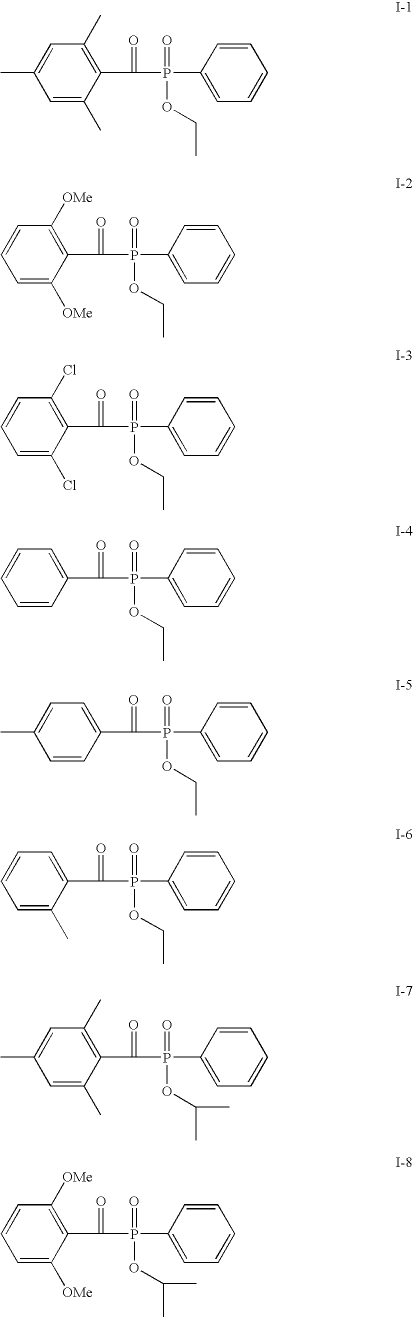

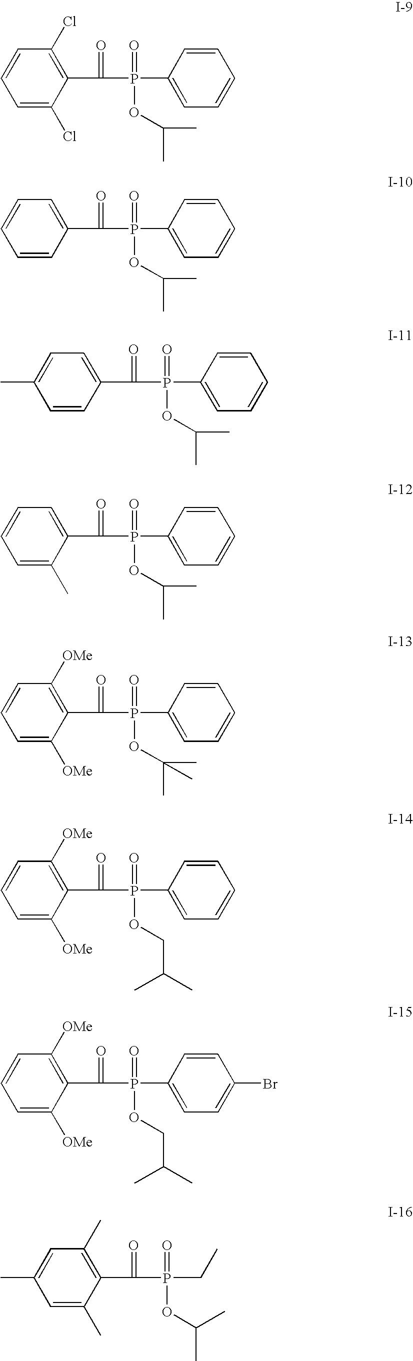

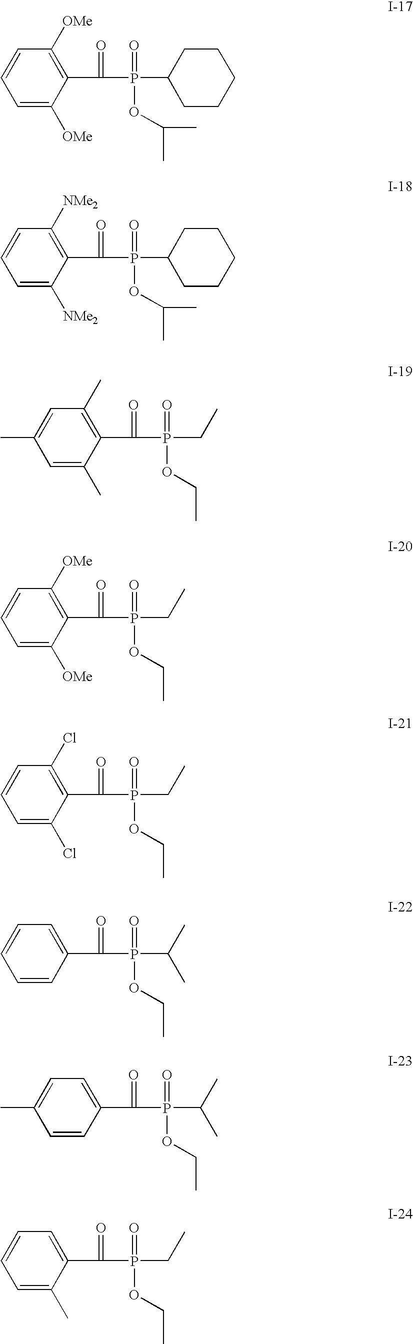

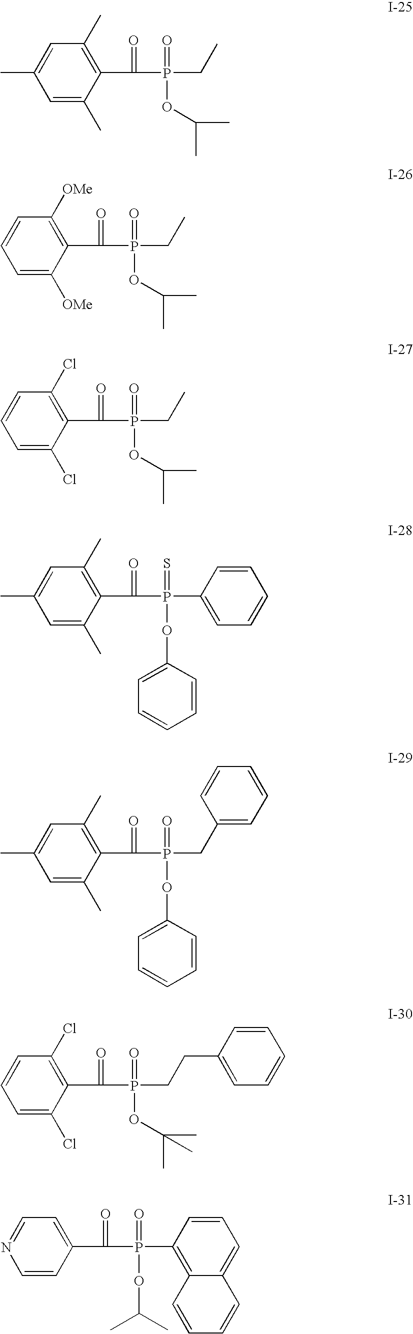

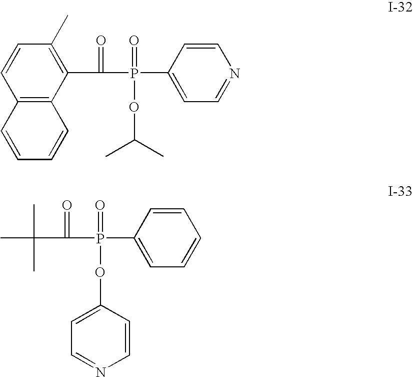

A method of synthesizing the above-described compound denoted by general formula (II) is described in detail in, for example, DE2830927A1, which is expressly incorporated herein by reference in its entirety. Examples described further below can also be referred to for synthesis methods.

Examples of cationic ring-opening photopolymerization initiators are 2,4,6-tris(trichloromethyl)-1,3,5-triazine, 2,4-bis(trichloromethyl)-6-(p-methoxyphenylvinyl)-1,3,5-triazine, diphenyliodonium tetrafluoroborate, 4,4′-di-t-butyldiphenyliodonium tetrafluoroborate, 4-diethylaminophenylbenzenediazonium hexafluorophosphate, and diphenyl-4-phenylthiophenylsulfonium hexafluorophosphate. These may be employed singly or in combinations of two or more. Sensitizing dyes, described further below, may be employed in combination in a manner in conformity with the wavelength of the light that is irradiated.

The content of the photo-induced polymerization initiator in the optical recording composition of the present invention is desirably 0.01 to 5 weight percent, preferably 1 to 3 weight percent. The content of equal to or greater than 0.01 weight percent can ensure an interference image of good sensitivity. The content of equal to or lower than 5 weight percent can permit the formation of a recording layer having adequate transmittance of the recording light and exhibiting good recording sensitivity.

The compound denoted by general formula (I) may be a monofunctional monomer having a single polymerizable group, or may be a polyfunctional monomer having two or more such groups per molecule. In the optical recording composition of the present invention, just a compound denoted by general formula (I) may be incorporated as a recording compound, or another polymerizable monomer may be incorporated along with the compound denoted by general formula (I). When another polymerizable monomer is incorporated along with the compound denoted by general formula (I), the quantity of the additional polymerizable monomer employed is desirably equal to or lower than 50 weight percent of the total polymerizable monomer.

Examples of additional monomers in the form of radical polymerizable monomers are: acryloylmorpholine, phenoxyethyl acrylate, isobornyl acrylate, 2-hydroxypropyl acrylate, 2-ethylhexyl acrylate, 1,6-hexanediol diacrylate, tripropyleneglycol diacrylate, neopentylglycol PO-modified diacrylate, 1,9-nonanediol diacrylate, hydroxypivalic acid neopentylglycol diacrylate, EO-modified bisphenol A diacrylate, polyethlyleneglycol diacrylate, pentaerythritol triacrylate, pentaerythritol tetraacrylate, pentaerythritol hexaacrylate, EO-modified glycerol triacrylate, trimethylolpropane triacrylate, EO-modified trimethylolpropane triacrylate, 2-naphtho-1-oxyethyl acrylate, 2-carbazoyl-9-ylethyl acrylate, (trimethylsilyloxy)dimethylsilylpropyl acrylate, vinyl-1-naphthoate, 2,4,6-tribromophenyl acrylate, pentabromoacrylate, phenylthioethyl acrylate, tetrahydrofurfuryl acrylate, bisphenoxyethanolfluorene diacrylate, styrene, p-chlorostyrene, N-vinylcarbazole, and N-vinylpyrrolidone. Of these, phenoxyethyl acrylate, 2,4,6-tribromophenyl acrylate, pentabromoacrylate, and bisphenoxyethanolfluorene diacrylate are desirable, and 2,4,6-tribromophenyl acrylate and bisphenoxyethanolfluorene diacrylate are preferred.

Examples of additional monomers in the form of cationic polymerizable monomers are: 2,3-epoxy-1-propane, 3,4-epoxy-1-butane, 1,6-hexanediol monoglycidyl ether, glycerol diglycidyl ether, glycerol propoxylate diglycidyl ether, glycidyl 4-hydroxyphenyl ether, glycidyl phenyl ether, 1,2-epoxyethylbenzene, bisphenol A diglycidyl ether, pentaerythritol tetra(3-ethyl-3-oxycetanylmethyl)ether, 3-ethylene carbonate, propylene carbonate, and γ-butyrolactone.

### Matrix

The recording layer of an optical recording medium normally comprises a polymer to hold the photopolymerization initiator and monomers related to the recording and storage, known as a matrix. The matrix can be employed for achieving enhanced coating properties, coating strength, and hologram recording characteristics. The optical recording composition of the present invention can comprise curing compounds in the form of a matrix binder and/or matrix forming components (matrix precursors). A method of forming the matrix by, for example, coating a composition containing the matrix precursor on the surface of a substrate and then curing it is desirable because it permits the formation of the recording layer without the use of, or using only a small quantity of, solvent. Thermosetting compounds and light-curing compounds employing catalysts and the like that cure when irradiated with light may be employed as these curing compounds. Thermosetting compounds are desirable from the perspective of recording characteristics.

The thermosetting compound suitable for use in the optical recording composition of the present invention is not specifically limited. The matrix contained in the recording layer may be suitably selected based on the objective. Examples are urethane resins formed from isocyanate compounds and alcohol compounds; epoxy compounds formed from oxysilane compounds; melamine compounds; formalin compounds; ester compounds of unsaturated acids such as (meth)acrylic acid and itaconic acid; and polymers obtained by polymerizing amide compounds.

Of these, polyurethane matrices formed from isocyanate compounds and alcohol compounds are preferable. From the perspective of recording retention properties, three-dimensional polyurethane matrices formed from polyfunctional isocyanates and polyfunctional alcohols are particularly preferred.

The details of polyfunctional isocyanates and polyfunctional alcohols capable of forming polyurethane matrices are described below.

Examples of the polyfunctional isocyanates are: biscyclohexylmethane diisocyanate, hexamethylene diisocyanate, phenylene-1,3-diisocyanate, phenylene-1,4-diisocyanate, 1-methoxyphenylene-2,4-diisocyanate, 1-methylphenylene-2,4-diisocyanate, 2,4-tolylene diisocyanate, 2,6-tolylene diisocyanate, 1,3-xylylene diisocyanate, 1,4-xylylene diisocyanate, biphenylene-4,4′-diisocyanate, 3,3′-dimethoxybiphenylene-4,4′-diisocyanate, 3,3′-dimethylbiphenylene-4,4′-diisocyanate, diphenylmethane-2,4′-diisocyanate, diphenylmethane-4,4′-diisocyanate, 3,3′-dimethoxydiphenylmethane-4,4′-diisocyanate, 3,3′-dimethyldiphenylmethane-4,4′-diisocyanate, naphthylene-1,5-diisocyanate, cyclobutylene-1,3-diisocyanate, cyclopentylene-1,3-diisocyanate, cyclohexylene-1,3-diisocyanate, cyclohexylene-1,4-diisocyanate, 1-methylcyclohexylene-2,4-diisocyanate, 1-methylcyclohexylene-2,6-diisocyanate, 1-isocyanate-3,3,5-trimethyl-5-isocyanate methylcyclohexane, cyclohexane-1,3-bis(methylisocyanate), cyclohexane-1,4-bis(methylisocyanate), isophorone diisocyanate, dicyclohexylmethane-2,4′-diisocyanate, dicyclohexylmethane-4,4′-diisocyanate, ethylene diisocyanate, tetramethylene-1,4-diisocyanate, hexamethylene-1,6-diisocyanate, dodecarnethylene-1,12-diisocyanate, phenyl-1,3,5-triisocyanate, diphenylmethane-2,4,4′-triisocyanate, diphenylmethane-2,5,4′-triisocyanate, triphenylmethane-2,4′,4″-triisocyanate, triphenylmethane-4,4′,4″-triisocyanate, diphenylmethane-2,4,2′,4′-tetraisocyanate, diphenylmethane-2,5,2′,5′-tetraisocyanate, cyclohexane-1,3,5-triisocyanate, cyclohexane-1,3,5-tris(methylisocyanate), 3,5-dimethylcyclohexane-1,3,5-tris(methylisocyanate), 1,3,5-trimethylcyclohexane-1,3,5-tris(methylisocyanate), dicyclohexylmethane-2,4,2′-triisocyanate, dicyclohexylmethane-2,4,4′-triisocyanate lysine isocyanate methyl ester, and prepolymers having isocyanates on both ends obtained by reacting a stoichiometrically excess quantity of one or more of these organic isocyanate compounds with a polyfunctional active hydrogen-containing compound. Of these, biscyclohexylmethane diisocyanate and hexamethylene diisocyanate are preferred. They may be employed singly or in combinations of two or more.

The polyfunctional alcohols may be in the form of a single polyfunctional alcohol, or in the form of a mixture with two or more polyfunctional alcohols. Examples of these polyfunctional alcohols are: glycols such as ethylene glycol, triethylene glycol, diethylene glycol, polyethylene glycol, propylene glycol, polypropylene glycol, and neopentyl glycol; diols such as butanediol, pentanediol, hexanediol, heptanediol, and tetramethylene glycol; bisphenols; compounds in the form of these polyfunctional alcohols modified by polyethyleneoxy chains or polypropyleneoxy chains; and compounds in the form of these polyfunctional alcohols modified by polyethyleneoxy chains or polypropyleneoxy chains, such as glycerin, trimethylolpropane, butanetriol, pentanetriol, hexanetriol, decanetriol, and other triols.

The content of the above-described matrix-forming components (or matrix) in the optical recording composition of the present invention is desirably 10 to 95 weight percent, preferably 35 to 90 weight percent. When the content is equal to or greater than 10 weight percent, stable interference images can be readily achieved. At equal to or less than 95 weight percent, desirable properties can be obtained from the perspective of diffraction efficiency.

### Other Components

Polymerization inhibitors and oxidation inhibitors may be added to the optical recording composition of the present invention to improve the storage stability of the optical recording composition, as needed.

Examples of polymerization inhibitors and oxidation inhibitors are: hydroquinone, p-benzoquinone, hydroquinone monomethyl ether, 2,6-ditert-butyl-p-cresol, 2,2′-methylenebis(4-methyl-6-tert-butylphenol), triphenylphosphite, trisnonylphenylphoshite, phenothiazine, and N-isopropyl-N′-phenyl-p-phenylenediamine.

The quantity of polymerization inhibitor or oxidation inhibitor added is preferably equal to or less than 3 weight percent of the total quantity of recording monomer. When the quantity added exceeds 3 weight percent, polymerization may slow down, and in extreme cases, ceases.

As needed, a sensitizing dye may be added to the optical recording composition of the present invention. Known compounds such as those described in “Research Disclosure, Vol. 200, 1980, December, Item 20036” and “Sensitizers” (pp. 160-163, Kodansha, ed. by K. Tokumaru and M. Okawara, 1987), which are expressly incorporated herein by reference in their entirety, and the like may be employed as sensitizing dyes.

Specific examples of sensitizing dyes are: 3-ketocoumarin compounds described in Japanese Unexamined Patent Publication (KOKAI) Showa No. 58-15603; thiopyrilium salt described in Japanese Unexamined Patent Publication (KOKAI) Showa No. 58-40302; naphthothiazole merocyanine compounds described in Japanese Examined Patent Publications (KOKOKU) Showa Nos. 59-28328 and 60-53300; and merocyanine compounds described in Japanese Examined Patent Publications (KOKOKU) Showa Nos. 61-9621 and 62-3842 and Japanese Unexamined Patent Publications (KOKAI) Showa Nos. 59-89303 and 60-60104, which are expressly incorporated herein by reference in their entirety.

Further examples are the dyes described in “The Chemistry of Functional Dyes” (1981, CMC Press, pp. 393-416) and “Coloring Materials” (60 [4] 212-224 (1987)), which are expressly incorporated herein by reference in their entirety. Specific examples are cationic methine dyes, cationic carbonium dyes, cationic quinoneimine dyes, cationic indoline dyes, and cationic styryl dyes.

Further, keto dyes such as coumarin (including ketocoumarin and sulfonocoumarin) dyes, merostyryl dyes, oxonol dyes, and hemioxonol dyes; nonketo dyes such as nonketo polymethine dyes, triarylmethane dyes, xanthene dyes, anthracene dyes, rhodamine dyes, acrylidine dyes, aniline dyes, and azo dyes; nonketo polymethine dyes such as azomethine dyes, cyanine dyes, carbocyanine dyes, dicarbocyanine dyes, tricarbocyanine dyes, hemicyanine dyes, and styryl dyes; and quinone imine dyes such as azine dyes, oxazine dyes, thiazine dyes, quinoline dyes, and thiazole dyes are included among the spectral sensitizing dyes.

These sensitizing dyes may be employed singly or in combinations of two or more.

A photo-heat converting material can be incorporated into the optical recording composition of the present invention for enhancing the sensitivity of the recording layer formed with the optical recording composition.

The photo-heat converting material is not specifically limited, and may be suitably selected based on the functions and properties desired. For example, for convenience during addition to the recording layer with the photopolymer and so as not to scatter incident light, an organic dye or pigment is desirable. From the perspectives of not absorbing and not scattering light from the light source employed in recording, infrared radiation-absorbing dyes are desirable.

Such infrared radiation-absorbing dyes are not specifically limited, and may be suitably selected based on the objective. However, cationic dyes, complex-forming dyes, quinone-based neutral dyes, and the like are suitable. The maximum absorption wavelength of the infrared radiation-absorbing dye preferably falls within a range of 600 to 1,000 nm, more preferably a range of 700 to 900 nm.

The content of infrared radiation-absorbing dye in the optical recording composition of the present invention can be determined based on the absorbance at the wavelength of maximum absorbance in the infrared region in the recording medium formed with the optical recording composition of the present invention. This absorbance preferably falls within a range of 0.1 to 2.5, more preferably a range of 0.2 to 2.0.

The optical recording composition of the present invention can be employed as various holographic recording compositions capable of recording information when irradiated with a light containing information. In particular, it is suited to use as a volume holographic recording composition. A recording layer can be formed by coating the optical recording composition of the present invention on a substrate, for example. When the optical recording composition of the present invention contains a thermosetting compound such as those set forth above, a matrix can be formed by promoting the curing reaction by heating following coating. The heating conditions can be determined based on the thermosetting resin employed. The recording layer can be formed by casting when the viscosity of the optical recording composition is adequately low. When the viscosity is so high that casting is difficult, a dispenser can be employed to spread a recording layer on a lower substrate, and an upper substrate pressed onto the recording layer so as to cover it and spread it over the entire surface, thereby forming a recording medium.

### Holographic Recording Medium

The holographic recording medium of the present invention comprises a recording layer comprising at least one compound denoted by general formula (I). The recording layer can be formed with the optical recording composition of the present invention. For example, the recording layer comprised of the optical recording composition of the present invention can be formed by the above-described method.

The recording layer of the holographic recording composition of the present invention comprises one or more of the compounds denoted by general formula (I). Since the compound denoted by general formula (I) has absorption characteristics that are suited to recording by irradiation with light of short wavelength, it is possible to form a holographic recording medium permitting highly sensitive, high-density recording in the short wavelength recording region. In the same manner as for the content in the optical recording composition of the present invention set forth above, the content of the compound denoted by general formula (I) in the recording layer is desirably 1 to 50 weight percent, preferably 1 to 30 weight percent, and more preferably, 2 to 10 weight percent. A content of equal to or lower than 50 weight percent can readily ensure a stable interference image, and a content of equal to or greater than 1 weight percent can yield desirable properties from the perspective of diffraction efficiency. The details of the various components in the recording layer of the holographic recording medium of the present invention are as set forth above for the optical recording composition of the present invention.

The holographic recording medium of the present invention is particularly suited to use as a holographic recording medium with a light source having a wavelength of around 400 nm. Since the holographic recording medium employs an incident diffraction light as a signal light, transmittance of the recording and reproducing lights is desirably high. For example, for a recording wavelength of 405 nm and a recording layer 500 μm in thickness, the addition of a polymerizable compound with a molecular weight of 400 in an amount of 10 weight percent relative to the matrix yields a concentration of about 0.018 mol/L. Considering the case where an initiator having a molar absorbance coefficient of about 80 mole·1·cm⁻¹ at 405 nm is added in a proportion of 15 molar percent relative to the quantity of the polymerizable compound, the transmittance of the recording layer is less than 60 percent when the molar absorbance coefficient of the polymerizable compound is equal to or greater than 200 mole·1·cm⁻¹. Since it is desirable for the transmittance of the recording medium to be equal to or greater than 60 percent, the molar absorbance coefficient of the polymerizable compound is desirably equal to or lower than 200 mole·1·cm⁻¹. As set forth above, the compounds denoted by general formula (I) are suitably employed as recording monomers in a holographic recording medium employing a light source with a wavelength of around 400 nm since they can achieve the above-described desirable absorption characteristics.

The holographic recording medium of the present invention comprises the above recording layer (holographic recording layer), and preferably comprises a lower substrate, a filter layer, a holographic recording layer, and an upper substrate. As needed, it may comprise additional layers such as a reflective layer, filter layer, first gap layer, and second gap layer.

The holographic recording medium of the present invention is capable of recording and reproducing information through utilization of the principle of the hologram. This may be a relatively thin planar hologram that records two-dimensional information or the like, or a volumetric hologram that records large quantities of information, such as three-dimensional images. It may be either of the transmitting or reflecting type. Since the holographic recording medium of the present invention is capable of recording high volumes of information, it is suitable for use as a volume holographic recording medium of which high recording density is demanded.

The method of recording a hologram on the holographic recording medium of the present invention is not specifically limited; examples are amplitude holograms, phase holograms, blazed holograms, and complex amplitude holograms. Among these, a preferred method is the so-called “collinear method” in which recording of information in volume holographic recording regions is carried out by irradiating an informing light and a reference light onto a volume holographic recording area as coaxial beams to record information by means of interference pattern through interference of the informing light and the reference light.

Details of substrates and various layers that can be incorporated into the holographic recording medium of the present invention will be described below.

### —Substrate—

The substrate is not specifically limited in terms of its shape, structure, size, or the like; these may be suitably selected based on the objective. For example, the substrate may be disk-shaped, card-shaped, or the like. A substrate of a material capable of ensuring the mechanical strength of the holographic recording medium can be suitably selected. When the light employed for recording and reproducing enters after passing through the substrate, a substrate that is adequately transparent at the wavelength region of the light employed is desirable.

Normally, glass, ceramic, resin, or the like is employed as the substrate material. From the perspectives of moldability and cost, resin is particularly suitable. Examples of such resins are: polycarbonate resin, acrylic resin, epoxy resin, polystyrene resin, acrylonitrile—styrene copolymers, polyethylene resin, polypropylene resin, silicone resin, fluorine resin, ABS resin, and urethane resin. Of these, from the perspective of moldability, optical characteristics, and cost, polycarbonate resin and acrylic resin are preferred. Synthesized resins and commercially available resins may both be employed as substrates.

Normally, address servo areas are provided on the substrate at prescribed angular intervals as multiple positioning areas extending linearly in a radial direction, with the fan-shaped intervals between adjacent address servo areas serving as data areas. Information for operating focus servos and tracking servos by the sampled servo method, as well as address information, is recorded (preformatted) as pre-embossed pits (servo pits) or the like in address servo areas. Focus servo operation can be conducted using the reflective surface of a reflective film. Wobble pits, for example, can be employed as information for operating a tracking servo. When the holographic recording medium is card-shaped, it is possible not to have a servo pit pattern.

The thickness of the substrate is not specifically limited, and may be suitably selected based on the objective: a thickness of 0.1 to 5 mm is preferable, with 0.3 to 2 mm being preferred. A substrate thickness of equal to or greater than 0.1 mm is capable of preventing shape deformation during disk storage, while a thickness of equal to or less than 5 mm can avoid an overall disk weight generating an excessive load on the drive motor.

### —Recording Layer—

The recording layer can be formed with the optical recording composition of the present invention and is capable of recording information by holography. The thickness of the recording layer is not specifically limited, and may be suitably selected based on the objective. A recording layer thickness falling within a range of 1 to 1,000 micrometers yields an adequate S/N ratio even when conducting 10 to 300 shift multiplexing, and a thickness falling within a range of 100 to 700 micrometers is advantageous in that it yields a markedly good S/N ratio.

### —Reflective Film—

A reflective film can be formed on the servo pit pattern surface of the substrate.

A material having high reflectance for the informing light and reference light is preferably employed as the material of the reflective film. When the wavelength of the light employed as the informing light and reference light ranges from 400 to 780 nm, examples of desirable materials are Al, Al alloys, Ag, and Ag alloys. When the wavelength of the light employed as the informing light and reference light is equal to or greater than 650 nm, examples of desirable materials are Al, Al alloys, Ag, Ag alloys, Au, Cu alloys, and TiN.

By employing an optical recording medium that reflects light as well as can be recorded and/or erased information such as a DVD (digital video disk) as a reflective film, it is possible to record and rewrite directory information, such as the areas in which holograms have been recorded, when rewriting was conducted, and the areas in which errors are present and for which alternate processing has been conducted, without affecting the hologram.

The method of forming the reflective film is not specifically limited and may be suitably selected based on the objective. Various vapor phase growth methods such as vacuum deposition, sputtering, plasma CVD, optical CVD, ion plating, and electron beam vapor deposition may be employed. Of these, sputtering is superior from the perspectives of mass production, film quality, and the like.

The thickness of the reflective film is preferably equal to or greater than 50 nm, more preferably equal to or greater than 100 nm, to obtain adequate reflectance.

### —Filter Layer—

A filter layer can be provided on the servo pits of the substrate, on the reflective layer, or on the first gap layer, described further below.

The filter layer has a function of reflecting selective wavelengths in which, among multiple light rays, only light of a specific wavelength is selectively reflected, permitting passing one light and reflecting a second light. It also has a function of preventing generation of noise in which irregular reflection of the informing light and the reference light by the reflective film of the recording medium is prevented without a shift in the selectively reflected wavelength even when the angle of incidence varies. Therefore, by stacking filter layers on the recording medium, it is possible to perform optical recording with high resolution and good diffraction efficiency.

The filter layer is not specifically limited and may be suitably selected based on the objective. For example, the filter layer can be comprised of a laminate in which at least one of a dichroic mirror layer, coloring material-containing layer, dielectric vapor deposition layer, single-layer or two- or more layer cholesteric layer and other layers suitably selected as needed is laminated. The thickness of the filter layer is not specifically limited and may be, for example, about 0.5 to 20 micrometers.

The filter layer may be laminated by direct application on the substrate or the like with the recording layer, or may be laminated on a base material such as a film to prepare a filter layer which is then laminated on the substrate.

### —First Gap Layer—

The first gap layer is formed as needed between the filter layer and the reflective film to flatten the surface of the lower substrate. It is also effective for adjusting the size of the hologram that is formed in the recording layer. That is, since the recording layer should form a certain size of the interference region of the recording-use reference light and the informing light, it is effective to provide a gap between the recording layer and the servo pit pattern.

For example, the first gap layer can be formed by applying a material such as an ultraviolet radiation-curing resin from above the servo pit pattern and curing it. When employing a filter layer formed by application on a transparent base material, the transparent base material can serve as the first gap layer.

The thickness of the first gap layer is not specifically limited, and can be suitably selected based on the objective. A thickness of 1 to 200 micrometers is desirable.

### —Second Gap Layer—

The second gap layer is provided as needed between the recording layer and the filter layer.

The material of the second gap layer is not specifically limited, and may be suitably selected based on the objective. Examples are: transparent resin films such as triacetyl cellulose (TAC), polycarbonate (PC), polyethylene terephthalate (PET), polystyrene (PS), polysulfone (PSF), polyvinylalcohol (PVA), and poly(methyl methacrylate) (PMMA); and norbornene resin films such as a product called ARTON film made by JSR Corporation and a product called Zeonoa made by Japan Zeon Co. Of these, those that are highly isotropic are desirable, with TAC, PC, the product called ARTON, and the product called Zeonoa being preferred.

The thickness of the second gap layer is not specifically limited and may be suitably selected based on the objective. A thickness of 1 to 200 micrometers is desirable.

Specific embodiments of the holographic recording medium of the present invention will be described in greater detail below. However, the present invention is not limited to these specific embodiments.

### First Implementation Embodiment

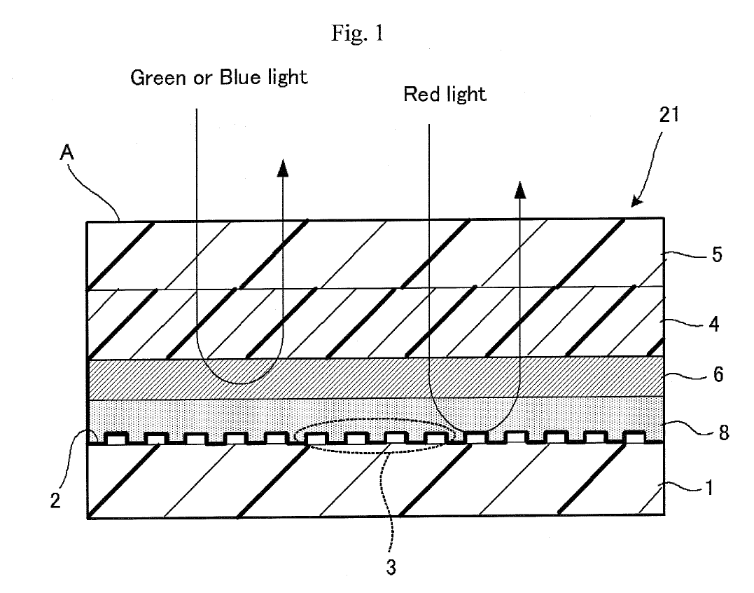

FIG. 1 is a schematic cross-sectional view of the configuration of the holographic recording medium according to the first implementation embodiment. In holographic recording medium 21 according to the first implementation embodiment, a servo pit pattern 3 is formed on substrate 1 made of polycarbonate resin or glass, and aluminum, gold, platinum, or the like is coated on servo pit pattern 3 to provide reflective film 2. In FIG. 1, servo pit pattern 3 has been formed over the entire surface of lower substrate 1, but the servo pit pattern may be formed cyclically. Servo pit pattern 3 is normally 1,750 Angstroms (175 nm) in height, and is quite small relative to the thickness of the substrate and the other layers.

First gap layer 8 is formed by spin coating or the like a material such as an ultraviolet radiation-curing resin on reflective film 2 of lower substrate 1. First gap layer 8 is effective for both the protection of reflective layer 2 and the adjustment of the size of the hologram formed in recording layer 4. That is, providing a gap between recording layer 4 and servo pit pattern 3 is effective for the formation of an interference area for the recording-use reference light and informing light of a certain size in recording layer 4.

Filter layer 6 is provided on first gap layer 8. Recording layer 4 is sandwiched between filter layer 6 and upper substrate 5 (a polycarbonate resin substrate or glass substrate) to form holographic recording medium 21.

FIG. 1 shows a filter layer 6 that passes only infrared radiation and blocks light of all other colors. Accordingly, since the informing light and recording and reproducing-use reference light are blue, they are blocked by filter layer 6 and do not reach reflective film 2. They return, exiting from entry and exit surface A.

Filter layer 6 is a multilayered vapor deposition film comprised of high refractive index layers and low refractive index layers deposited in alternating fashion.

Filter layer 6, comprised of a multilayered vapor deposition film, may be formed directly on first gap layer 8 by vacuum vapor deposition, or a film comprised of a multilayered vapor deposition film formed on a base material may be punched into the shape of a holographic recording medium to employed as filter layer 6.

In this embodiment, holographic recording medium 21 may be disk-shaped or card-shaped. When card-shaped, the servo pit pattern may be absent. In holographic recording medium 21, the lower substrate is 0.6 mm, first gap layer 8 is 100 micrometers, filter layer 6 is 2 to 3 micrometers, recording layer 4 is 0.6 mm, and upper substrate 5 is 0.6 mm in thickness, for a total thickness of about 1.9 mm.

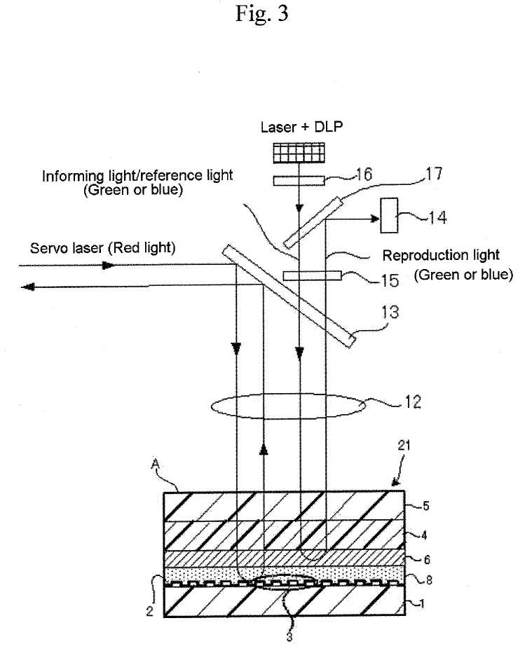

An optical system applicable for the recording of information on and the reproduction of information from holographic recording medium 21 will be described with reference to FIG. 3.

First, a light (red light) emitted by a servo laser is nearly 100 percent reflected by dichroic mirror 13, passing through objective lens 12. Objective lens 12 directs the servo light onto holographic recording medium 21 so that it focuses at a point on reflective film 2. That is, dichroic mirror 13 passes light of green and blue wavelengths while reflecting nearly 100 percent of red light. The servo light entering entry and exit surface A to which and from which the light enters and exits of holographic recording medium 21 passes through upper substrate 5, recording layer 4, filter layer 6, and first gap layer 8, is reflected by reflective layer 2, and passes back through first gap layer 8, filter layer 6, recording layer 4, and upper substrate 5, exiting entry and exit surface A. The returning light that exits passes through objective lens 12, is nearly 100 percent reflected by dichroic mirror 13, and the servo information is detected by a servo information detector (not shown in FIG. 3). The servo information that is detected is employed for focus servo, tracking servo, slide servo, and the like. When the hologram material included in recording layer 4 is not sensitive to red light, the servo light passes through recording layer 4 without affecting recording layer 4, even when the servo light is randomly reflected by reflective film 2. Since the light in the form of the servo light reflected by reflective film 2 is nearly 100 percent reflected by dichroic mirror 13, the servo light is not detected by a CMOS sensor or CCD 14 for reproduction image detection and thus does not constitute noise to the reproduction light.

The informing light and recording-use reference light generated by the recording/reproducing laser passes through polarizing plate 16 and is linearly polarized. It then passes through half mirror 17, becoming circularly polarized light at the point where it passes through ¼ wavelength plate 15. The light then passes through dichroic mirror 13, and is directed by objective lens 12 onto holographic recording medium 21 so that the informing light and recording-use reference light form an interference pattern in recording layer 4. The informing light and recording-use reference light enter through entry and exit surface A, interfering with each other to form an interference pattern in recording layer 4. Subsequently, the informing light and recording-use reference light pass through recording layer 4, entering filter layer 6. However, they are reflected before reaching the bottom surface of filter layer 6, returning. That is, neither the informing light nor the recording-use reference light reaches reflective film 2. That is because filter layer 6 is a multilayered vapor deposition layer in which multiple high refractive index and low refractive index layers are alternatively laminated, and has the property of passing only red light.

### Second Implementation Embodiment

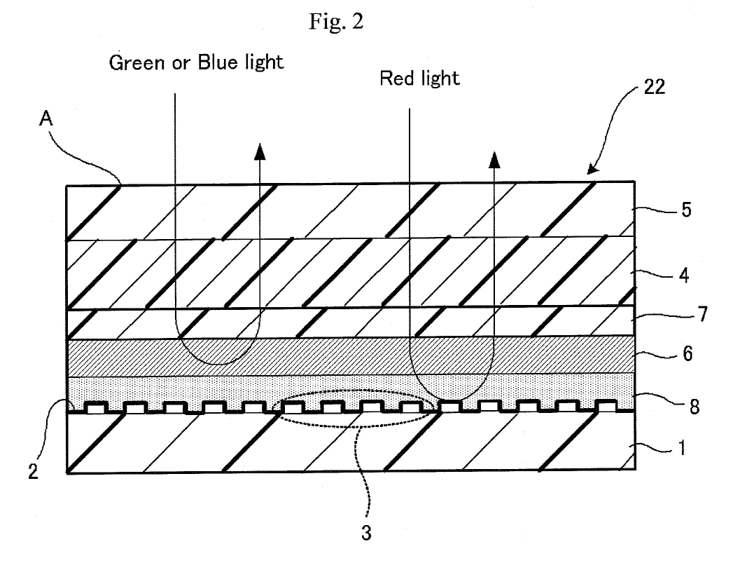

FIG. 2 is a schematic cross-sectional view of the configuration of the holographic recording medium according to the second implementation embodiment. A servo pit pattern 3 is formed on substrate 1 made of polycarbonate resin or glass in the holographic recording medium 22 accoding to the second implementation embodiment. Reflective film 2 is provided by coating aluminum, gold, platinum, or the like on the surface of servo pit pattern 3. Servo pit pattern 3 is normally 1,750 Angstroms (175 nm) in height in the same manner as in the first implementation embodiment.

The configuration of the second implementation embodiment differs from that of the first implementation embodiment in that second gap layer 7 is provided between filter layer 6 and recording layer 4 in holographic recording medium 22 according to the second implementation embodiment. A point at which the informing light and reproduction light are focused is present in second gap layer 7. When this area is embedded in a photopolymer, excessive consumption of monomer occurs due to excess exposure, and multiplexing recording capability diminishes. Accordingly, it is effective to provide a nonreactive transparent second gap layer.

Filter layer 6 in the form of a multilayered vapor deposition film comprised of multiple layers in which multiple high refractive index and low refractive index layers are alternately laminated is formed over first gap layer 8 once first gap layer 8 has been formed, and the same one as employed in the first implementation embodiment can be employed as filter layer 6 in the second implementation embodiment.

In holographic recording medium 22 of the second implementation embodiment, lower substrate 1 is 1.0 mm, first gap 8 is 100 micrometers, filter layer 6 is 3 to 5 micrometers, second gap layer 7 is 70 micrometers, recording layer 4 is 0.6 mm, and upper substrate 5 is 0.4 mm in thickness, for a total thickness of about 2.2 mm.

When recording or reproducing information, a red servo light and a green informing light and recording/reproducing reference light are directed onto holographic recording medium 22 of the second implementation embodiment having the configuration set forth above. The servo light enters through entry and exit surface A, passing through recording layer 4, second gap layer 7, filter layer 6, and first gap layer 8, and is reflected by reflective film 2, returning. The returning light then passes sequentially back through first gap layer 8, filter layer 6, second gap layer 7, recording layer 4, and upper substrate 5, exiting through entry and exit surface A. The returning light that exits is used for focus servo, tracking servo, and the like. When the hologram material included in recording layer 4 is not sensitive to red light, the servo light passes through recording layer 4 and is randomly reflected by reflective film 2 without affecting recording layer 4. The green informing light and the like enters through entry and exit surface A, passing through recording layer 4 and second gap layer 7, and is reflected by filter layer 6, returning. The returning light then passes sequentially back through second gap layer 7, recording layer 4, and upper substrate 5, exiting through entry and exit layer A. During reproduction, as well, the reproduction-use reference light and the reproduction light generated by irradiating the reproduction-use reference light onto recording layer 4 exit through entry and exit surface A without reaching reflective film 2. The optical action around holographic recording medium 22 (objective lens 12, filter layer 6, and detectors in the form of CMOS sensors or CCD 14 in FIG. 3) is identical to that in the first implementation embodiment and thus the description thereof is omitted.

The method of recording information on the holographic recording medium of the present invention will be described below.

An interference image can be formed on the recording layer of the holographic recording medium of the present invention by irradiation of an informing light and a reference light to the recording layer, and a fixing light can be irradiated to the recording layer on which the interference image has been formed to fix the interference image.

A light having coherent properties can be employed as the informing light. By irradiating the informing light and reference light onto the recording medium so that the optical axes of the informing light and reference light are coaxial, it is possible to record in the recording layer an interference image generated by interference of the informing light and reference light. Specifically, a informing light imparted with a two dimensional intensity distribution and a reference light of intensity nearly identical to that of the informing light are superposed in the recording layer and the interference pattern that they form is used to generate an optical characteristic distribution in the recording layer, thereby recording information. The wavelengths of the informing light and reference light are preferably equal to or greater than 400 nm, more preferably 400 to 2,000 nm, and further preferably, 400 to 700 nm.

After recording information (forming an interference image) by irradiating the informing light and reference light, a fixing light can be irradiated to fix the interference image. The wavelength of the fixing light is preferably less than 400 nm, more preferably equal to or greater than 100 nm but less than 400 nm, and further preferably, equal to or greater than 200 nm but less than 400 nm.

Information can be reproduced by irradiating a reference light onto an interference image formed by the above-described method. In the course of reading (reproducing) information that has been written, just a reference light is irradiated onto the recording layer with the same arrangement as during recording, causing a reproduction light having an intensity distribution corresponding to the optical characteristic distribution formed in the recording layer to exit the recording layer.

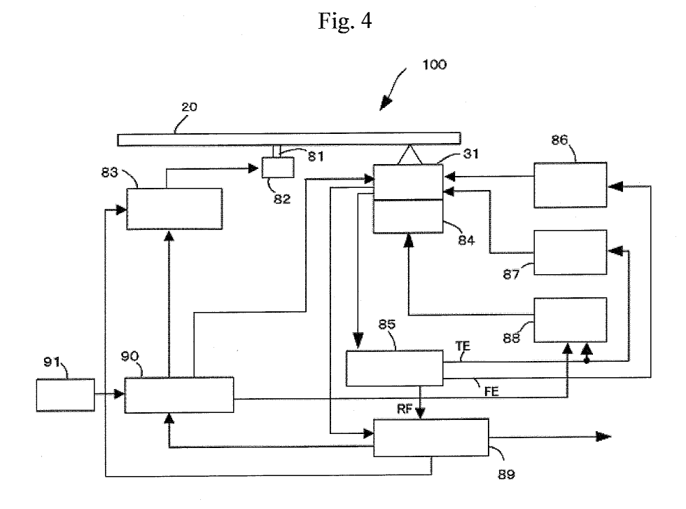

An optical recording and reproducing device suited to use in the recording and reproducing of information in the holographic recording medium of the present invention will be described with reference to FIG. 4.

The optical recording and reproducing device 100 of FIG. 4 is equipped with spindle 81 on which is mounted holographic recording medium 20, spindle motor 82 rotating spindle 81, and spindle servo circuit 83 controlling spindle motor 82 so that it maintains holographic recording medium 20 at a prescribed rpm level.

Recording and reproducing device 100 is further equipped with pickup 31 for recording information by irradiating a informing light and a recording-use reference light onto holographic recording medium 20, and for reproducing information that has been recorded on holographic recording medium 20 by irradiating a reproducing-use reference light onto holographic recording medium 20 and detecting the reproduction light; and driving device 84 capable of moving pickup 31 radially with respect to holographic recording medium 20.

Optical recording and reproducing device 100 is equipped with detection circuit 85 for detecting focus error signal FE, tracking error signal TE, and reproduction signal RF based on the output signals of pickup 31; focus servo circuit 86 that operates a focus servo by driving an actuator in pickup 31 to move an objective lens (not shown in FIG. 4) in the direction of thickness of holographic recording medium 20 based on focus error signal FE detected by detection circuit 85; tracking servo circuit 87 that operates a tracking servo by driving an actuator in pickup 31 to move an objective lens in the radial direction of holographic recording medium 20 based on tracking error signal TE detected by detection circuit 85; and slide servo circuit 88 that operates a slide servo by controlling drive device 84 to move pickup 31 in the radial direction of holographic recording medium 20 based on instructions from a controller, described further below, and tracking error signal TE.

Optical recording and reproducing device 100 is further equipped with signal processing circuit 89 that decodes the output data of a CCD array or CMOS in pickup 31 to reproduce data recorded in the data areas of holographic recording medium 20, reproduces a base clock based on reproduction signal RF from detection circuit 85, and determines addresses; controller 90 that effects overall control of optical recording and reproducing device 100; and operation element 91 providing various instructions to controller 90. Controller 90 inputs the base clock and address information outputted by signal processing circuit 89 and controls pickup 31, spindle servo circuit 83, slide servo circuit 88, and the like. Spindle servo circuit 83 inputs the base clock that is outputted by signal processing circuit 89. Controller 90 comprises a central processing unit (CPU), read only memory (ROM), and random access memory (RAM). The functions of controller 90 can be realized by having the CPU that employs the RAM as a work area and execute programs stored in the ROM.

### EXAMPLES

The present invention will be described in detail below based on examples. However, the present invention is not limited to the examples.

### Synthesis Example 1

Example Compound SM-6 was synthesized by the following scheme according to Synthesis Scheme 1 set forth above.

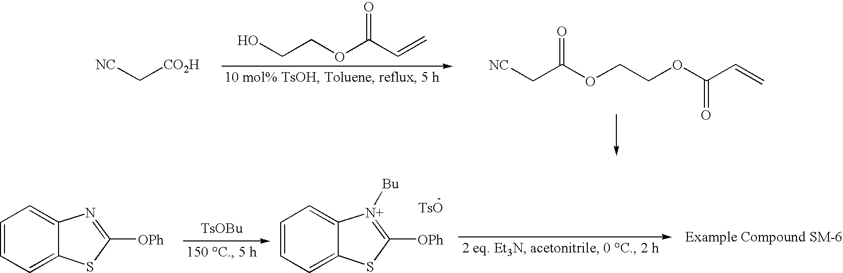

Identification results are given below:

¹H NMR (300 MHz, CDCl₃) δ1.09 (t, 3H), 1.52 (q, 2H), 1.89-2.00 (m, 2H), 4.43-4.53 (m, 4H), 4.62 (t, 2H), 5.84 (d, 1H), 6.18 (dd, 2H), 6.43 (d, 1H), 7.28-7.35 (m, 2H), 7.46 (t, 1H), 7.65 (d, 1H).

### Synthesis Example 2

Example Compound SM-37 was synthesized according to Synthesis Scheme 1 in the same manner as in Example 1. The identification results are given below:

### ¹H NMR (300 MHz, CDCl₃) δ0.99 (t, 3H), 1.48 (m, 2H), 1.78-1.99 (m, 6H), 4.23 (m, 4H), 4.40 (t, 2H), 5.81 (d, 1H), 6.13 (dd, 2H), 6.40 (d, 1H), 7.18-7.40 (m, 3H), 7.49 (d, 1H).

### Synthesis Example 3

Example Compound SM-16 was synthesized by the following scheme according to Synthesis Scheme 2 set forth above.

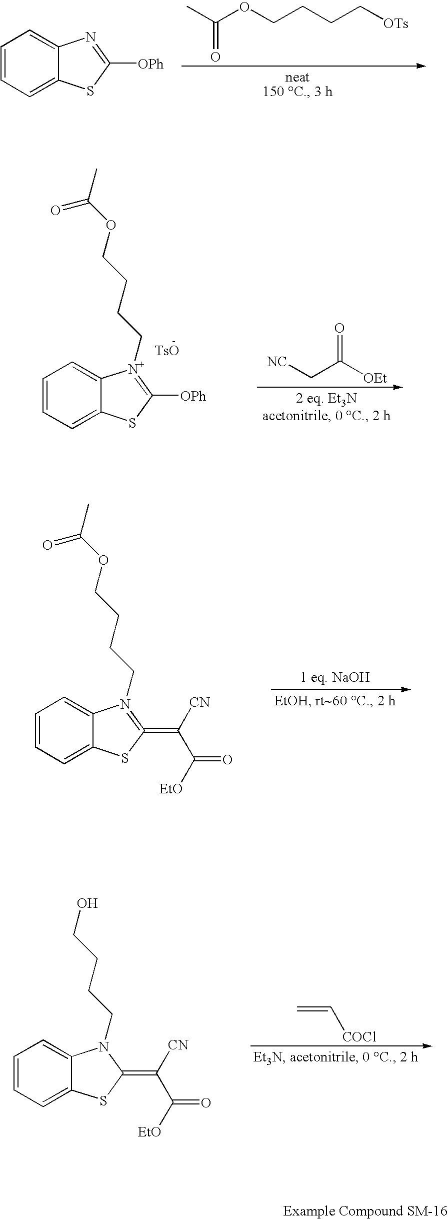

Identification results are given below:

### ¹H NMR (300 MHz, CDCl₃) δ1.38 (t, 3H), 1.89-1.99 (m, 2H), 2.01-2.12 (m, 2H), 4.22-4.38 (m, 4H), 4.64 (t, 2H), 5.87 (d, 1H), 6.17 (dd, 2H), 6.42 (d, 1H), 7.28-7.37 (m, 2H), 7.47 (t, 1H), 7.66 (d, 1H)

### Synthesis Example 4

Example Compound SM-4 was synthesized according to Synthesis Scheme 1 set forth above. The identification results are given below:

### ¹H NMR (300 MHz, CDCl₃) δ1.01(t, 3H), 1.51(m, 2H), 1.8-2.0(m, 6H), 4.22(m, 2H), 4.30(m, 2H), 4.58(t, 2H), 5.81(d, 1H), 6.11(dd, 1H), 6.41(d, 1H), 7.20(d, 1H), 7.40(d, 1H), 7.61(d, 1H).

### Synthesis Example 5

Example Compound SM-5 was synthesized according to Synthesis Scheme 1 set forth above. The identification results are given below:

### ¹H NMR (300 MHz, CDCl₃) δ1.01(t, 3H), 1.51(m, 2H), 1.8-2.0(m, 6H), 3.88(s, 3H), 4.21(m, 2H), 4.29(m, 2H), 4.56(t, 2H), 5.81(d, 1H), 6.12(dd, 1H), 6.41(d, 1H), 7.01(dd, 1H), 7.12(d, 1H), 7.20(d, 1H).

### Synthesis Example 6

Example Compound SM-63 was synthesized by the following scheme based on Synthesis Scheme 1 set forth above.

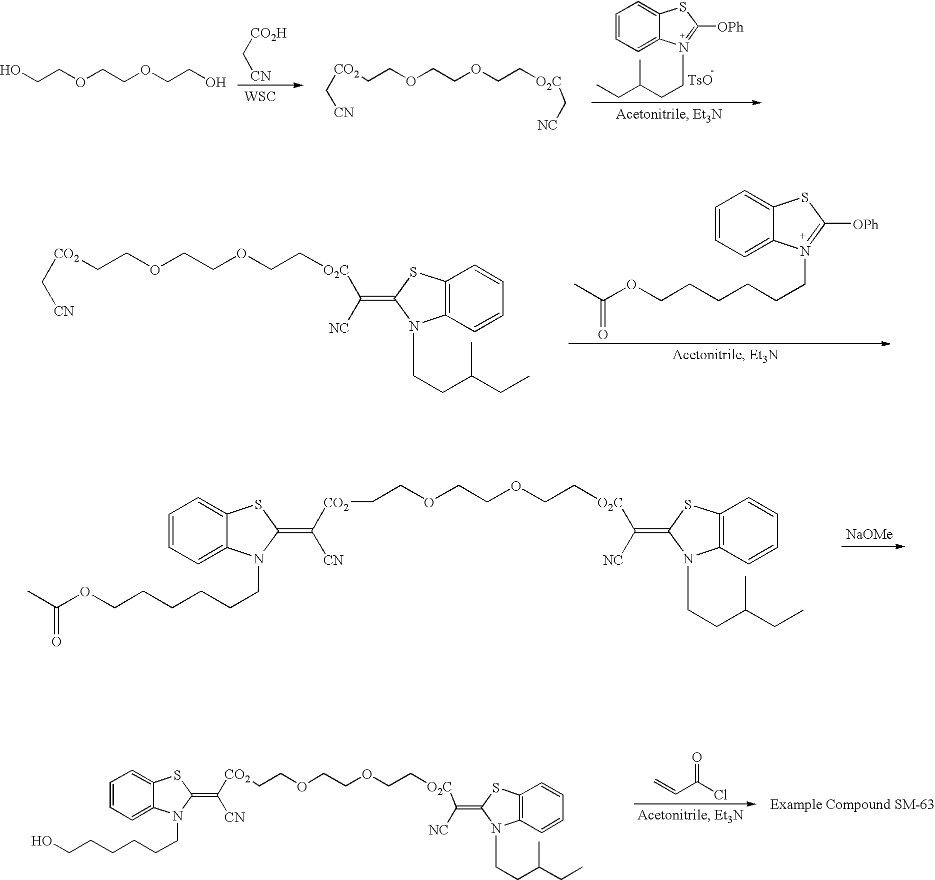

The identification results are given below:

¹H NMR (300 MHz, CDCl₃) δ0.92(t, 3H), 1.05(d, 3H), 1.30(m, 1H), 1.4˜1.8(m, 10H), 1.95(m, 2H), 3.77(s, 4H), 3.86(t, 4H), 4.17(t, 2H), 4.42(t, 4H), 4.60(m, 4H), 5.81(d, 1H), 6.11(dd, 1H), 6.40(d, 1H), 7.28(m, 4H), 7.43(t, 2H), 7.60(d, 2H).

### Synthesis Example 7

Example Compound SM-64 was synthesized by the following scheme based on Synthesis Scheme 1 set forth above.

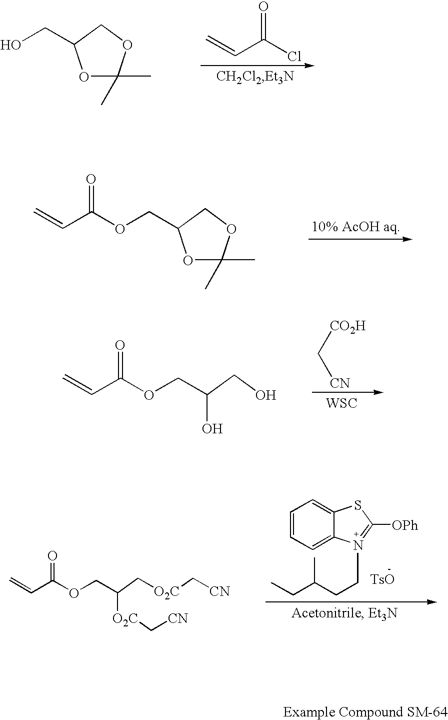

The identification results are given below:

### ¹H NMR (300 MHz, CDCl₃) δ0.91(m, 6H), 1.02(m, 6H), 1.28(m, 2H), 1.46(m, 2H), 1.70(m, 4H), 1.93(m, 2H), 4.45(m, 2H), 4.62(m, 6H), 5.54(m, 1H), 5.86(d, 1H), 6.18(dd, 1H), 6.47(d, 1H), 7.30(m, 4H), 7.46(t, 2H), 7.64(d, 2H).

### Synthesis Example 8

Example Compound SM-67 was synthesized by the following scheme based on Synthesis Scheme 1 set forth above.

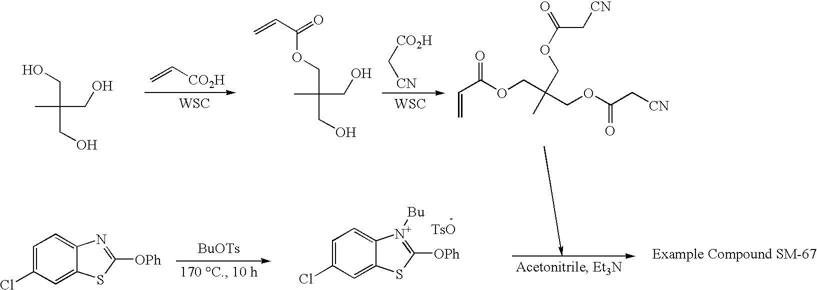

The identification results are given below:

### ¹H NMR (300 MHz, CDCl₃) δ1.02(t, 6H), 1.21(s, 3H), 1.54(m, 4H), 1.91(m, 4H), 4.30(s, 4H), 4.56(t, 4H), 5.83(d, 1H), 6.16(dd, 1H), 6.42(d, 1H), 7.20(d, 2H), 7.40(dd, 2H), 7.59(d, 2H).

### Synthesis Example 9

Example Compound SM-69 was synthesized by the following scheme based on Synthesis Scheme 1 set forth above.

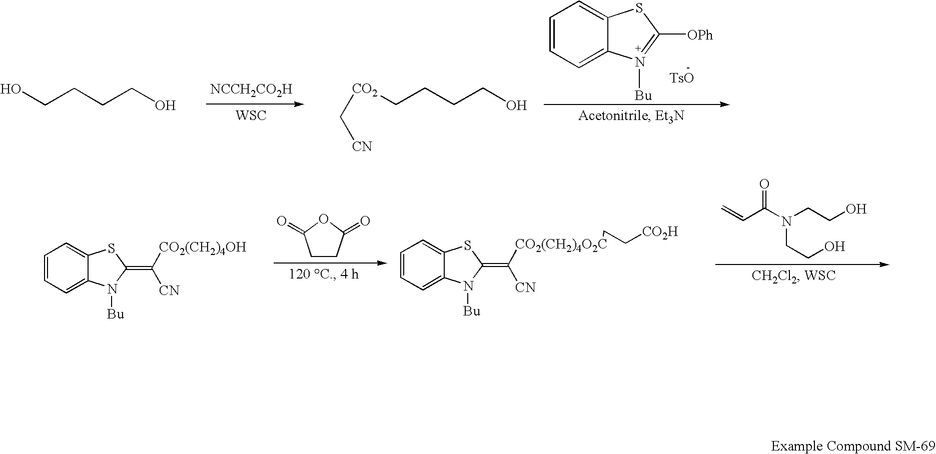

The identification results are given below:

### ¹H NMR (400 MHz, CDCl₃) δ1.01(t, 6H), 1.56(m, 4H), 1.92(m, 4H), 2.62(m, 4H), 2.69(m, 4H), 3.69(m, 4H), 3.81(m, 8H), 4.27(m, 8H), 4.41(t, 4H), 4.60(t, 4H), 5.72(d, 1H), 6.38(d, 1H), 6.62(dd, 1H), 7.31(m, 4H), 7.47(t, 2H), 7.66(d, 1H)

Absorption Measurement

The absorption spectra of solutions of the Example Compounds synthesized in Synthesis Examples 1 to 9 and Comparative Compounds 1 to 5 below (solvent: methylene chloride, solution concentration: 1×10⁻⁵ mol/L) were measured with a UV-3600 (made by Shimadzu Corporation), and the molar absorbance coefficient (ε) was calculated from the measured absorbance values obtained. The maximum absorption wavelength λ max and the molar absorbance coefficient at λ max were obtained from the absorption spectra. The results are given in Table 1.

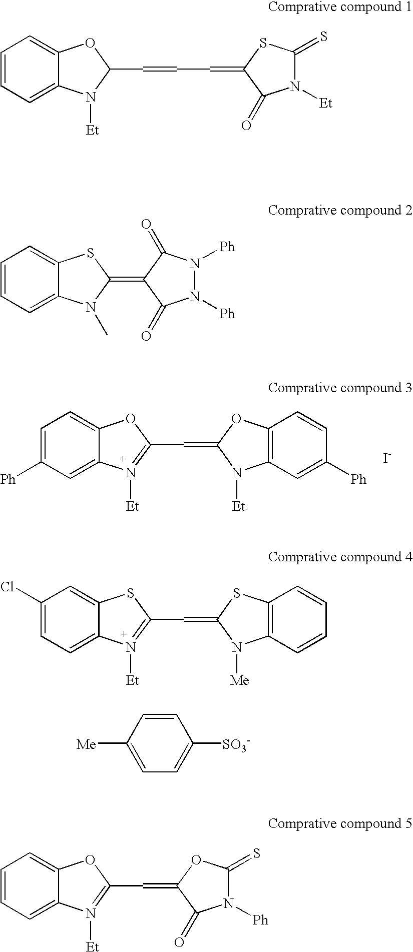

Comparative compound 1 is a dye moiety of Compound DM-14 described in Japanese Unexamined Patent Publication (KOKAI) No. 2005-275158. Comparative compound 2 is a dye moiety of Compound No. 1-1 described in Japanese Unexamined Patent Publication (KOKAI) No. 2007-272044. Comparative compound 3 is a dye moiety of Compound No. 1-2 described in Japanese Unexamined Patent Publication (KOKAI) No. 2007-272044. Comparative compound 4 is a dye moiety of Compound No. 1-3 described in Japanese Unexamined Patent Publication (KOKAI) No. 2007-272044. Comparative compound 5 is a dye moiety of Compound No. 1-5 described in Japanese Unexamined Patent Publication (KOKAI) No. 2007-272044.

TABLE 1

| | λmax | ε at λmax | ε at 405 nm |
|---|---|---|---|
| | (nm) | (mole · l · cm⁻¹) | (mole · l · cm⁻¹) |
| Ex. Compound SM-6 | 341 | 41,500 | ~0 |
| Ex. Compound SM-16 | 341 | 39,300 | ~0 |
| Ex. Compound SM-37 | 318 | 38,000 | ~0 |
| Ex. Compound SM-4 | 345 | 47,400 | ~0 |
| Ex. Compound SM-5 | 349 | 39,600 | ~0 |
| Ex. Compound SM-63 | 341 | 73,000 | ~0 |
| Ex. Compound SM-64 | 343 | 78,000 | ~0 |
| Ex. Compound SM-67 | 347 | 88,800 | ~0 |
| Ex. Compound SM-69 | 341 | 82,400 | ~0 |
| Comp. Compound 1 | 492 | 98,700 | 2260 |
| Comp. Compound 2 | 378 | 43,900 | 7700 |
| Comp. Compound 3 | 387 | 100,100 | 2940 |
| Comp. Compound 4 | 378 | 49,800 | 570 |
| Comp. Compound 5 | 368 | 36,200 | 300 |

It will be understood from Table 1 that the value of ε at 405 nm for Example Compounds SM-6, SM-16, SM-37, SM-4, SM-5, SM-63, SM-64, SM-67, and SM-69 was about 0 mole·1·cm⁻¹, indicating almost no absorption. The value of λ max was less than 405 nm, which was suitable for recording and reproduction with a laser in the 405 nm region.

### Synthesis Example 10

### Examples of Synthesizing Compounds Denoted by General Formula (II)

Example Compounds (I-2), (I-3), (I-8), and (I-9) were synthesized by the general scheme given below based on the method described in DE2830927A1. In the following scheme, R¹¹ to R¹³ have the same definitions as in general formula (II). Various compounds in which R¹¹ to R¹³ vary can be synthesized by the following scheme by employing different starting materials in synthesis.

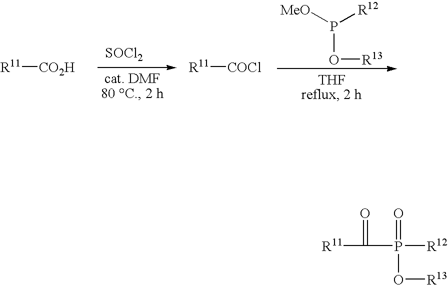

The identification results of Example Compounds (I-2), (I-3), (I-8) and (I-9) thus obtained are given below.

### <I-2>

### ¹H NMR (300 MHz, CDCl₃) δ1.32 (t, 3H), 3.62(s, 6H), 4.13-4.26 (m, 2H), 6.49 (d, 2H), 7.32(t, 1H), 7.40-7.51 (m, 2H), 7.54-7.59(m, 1H), 7.79 (dd, 2H)

### <I-3>

### ¹H NMR (300 MHz, CDCl₃) δ1.37 (d, 3H), 1.39 (d, 3H), 4.91-4.98 (m, 1), 7.29 (s, 3H), 7.47-7.51 (m, 2H), 7.59-7.61 (m, 1H), 7.91 (dd, 2H)

<1-8>

### ¹H NMR (300 MHz, CDCl₃) δ1.34 (d, 3H), 1.38 (d, 3H), 3.67(s, 6H), 4.68-4.80 (m, 1H), 7.32 (t, 1H), 7.41-7.50 (m, 2H), 7.52-7.59 (m, 1H), 7.90 (dd, 2H)

### <I-9>

### ¹H NMR (300 MHz, CDCl₃) δ1.36 (t, 3H), 4.41 (q, 2H), 7.28 (s, 3H), 7.58-7.64 (m, 1H), 7.93 (dd, 2H)

### Example 1

### Preparation of Holographic Recording Composition

A 6.4 g quantity of hexamethylene diisocyanate (made by Mitsui Chemicals Polyurethanes, Inc.; trade name: Takenate T-700), 5.21 g of polypropylene oxide triol (made by Mitsui Chemicals Polyurethanes, Inc.; trade name: MN-300), 4.64 g of polyethylene glycol (made by Tokyo Chemical Industry Co., Ltd.), 1.85 g of Example Compound SM-6, 0.16 g of photo-induced polymerization initiator (2,4,6-trimethylbenzoylphenylphosphinic acid ethyl ester; trade name: Lucirin TPO-L, made by BASF Japan), and 0.20 g of amine curing catalyst (made by SAN-APRO; trade name: U-CAT 410) were mixed under a nitrogen gas flow to prepare a holographic recording composition.

### Example 2

### Preparation of Holographic Recording Composition

With the exception that Example Compound SM-6 in Example 1 was replaced with 1.85 g of Example Compound SM-16, a holographic recording composition was prepared in the same manner as in Example 1.

### Example 3

### Preparation of Holographic Recording Composition

With the exception that Example Compound SM-6 in Example 1 was replaced with 1.85 g of Example Compound SM-37, a holographic recording composition was prepared in the same manner as in Example 1.

### Example 4

### Preparation of Holographic Recording Composition

With the exception that the 0.16 g of photo-induced polymerization initiator (2,4,6-trimethylbenzoylphenylphosphinic acid ethyl ester; trade name: Lucirin TPO-L, made by BASF Japan) in Example 1 was replaced with 0.16 g of Example Compound I-8, a holographic recording composition was prepared in the same manner as in Example 1.

### Example 5

### Preparation of Holographic Recording Composition

With the exception that the 0.16 g of photo-induced polymerization initiator (2,4,6-trimethylbenzoylphenylphosphinic acid ethyl ester; trade name: Lucirin TPO-L, made by BASF Japan) in Example 2 was replaced with 0.16 g of Example Compound I-8, a holographic recording composition was prepared in the same manner as in Example 2.

### Example 6

### Preparation of Holographic Recording Composition

With the exception that the 0.16 g of photo-induced polymerization initiator (2,4,6-trimethylbenzoylphenylphosphinic acid ethyl ester; trade name: Lucirin TPO-L, made by BASF Japan) in Example 3 was replaced with 0.16 g of Example Compound I-8, a holographic recording composition was prepared in the same manner as in Example 3.

### Example 7

### Preparation of Holographic Recording Composition

With the exception that Example Compound SM-6 in Example 4 was replaced with 1.85 g of Example Compound SM-4, a holographic recording composition was prepared in the same manner as in Example 4.

### Example 8

### Preparation of Holographic Recording Composition

With the exception that Example Compound SM-6 in Example 4 was replaced with 1.85 g of Example Compound SM-5, a holographic recording composition was prepared in the same manner as in Example 4.

### Example 9

### Preparation of Holographic Recording Composition

With the exception that Example Compound SM-6 in Example 4 was replaced with 1.85 g of Example Compound SM-63, a holographic recording composition was prepared in the same manner as in Example 4.

### Example 10

### Preparation of Holographic Recording Composition

With the exception that Example Compound SM-6 in Example 4 was replaced with 1.85 g of Example Compound SM-64, a holographic recording composition was prepared in the same manner as in Example 4.

### Example 11

### Preparation of Holographic Recording Composition

With the exception that Example Compound SM-6 in Example 4 was replaced with 1.85 g of Example Compound SM-67, a holographic recording composition was prepared in the same manner as in Example 4.

### Example 12

### Preparation of Holographic Recording Composition

With the exception that Example Compound SM-6 in Example 4 was replaced with 1.85 g of Example Compound SM-69, a holographic recording composition was prepared in the same manner as in Example 4.

### Comparative Example 1

### Preparation of Holographic Recording Composition

A 6.4 g quantity of hexamethylene diisocyanate (made by Mitsui Chemicals Polyurethanes, Inc.; trade name: Takenate T-700), 5.21 g of polypropylene oxide triol (made by Mitsui Chemicals Polyurethanes, Inc.; trade name: MN-300), 4.64 g of polyethylene glycol (made by Tokyo Chemical Industry Co., Ltd.), 1.85 g of 2,4,6-tribromophenyl acrylate (Dai-ichi Kogyo Seiyaku Co., Ltd.; trade name BR-30), 0.16 g of photo-induced polymerization initiator (2,4,6-trimethylbenzoylphenyl-phosphinic acid ethyl ester; trade name: Lucirin TPO-L, made by BASF Japan), and 0.20 g of amine curing catalyst (made by SAN-APRO; trade name: U-CAT 410) were mixed under a nitrogen gas flow to prepare a holographic recording composition.

### Comparative Example 2

### Preparation of Holographic Recording Composition

With the exception that the 1.85 g of 2,4,6-tribromophenyl acrylate (Dai-ichi Kogyo Seiyaku Co., Ltd.; trade name BR-30) employed in Comparative Example 1 was replaced with 1.85 g of the following monomer (R-1) (λ max=426 nm (ε=53100) in CH₂Cl₂, ε_{at 405 nm}=36100) described in Japanese Unexamined Patent Publication (KOKAI) No. 2005-275158, a holographic recording composition was prepared in the same manner as in Comparative Example 1.

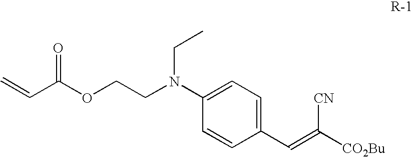

### Comparative Example 3

### Preparation of Holographic Recording Composition

With the exception that the 1.85 g of 2,4,6-tribromophenyl acrylate (Dai-ichi Kogyo Seiyaku Co., Ltd.; trade name BR-30) employed in Comparative Example 1 was replaced with 1.85 g of the following monomer (R-2) (λ max 460 nm (ε=68000) in CH₂Cl₂, ε_{at 405 nm}=45200) described in Japanese Unexamined Patent Publication (KOKAI) No. 2007-272044, a holographic recording composition was prepared in the same manner as in Comparative Example 1.

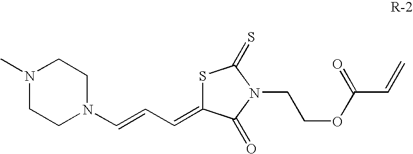

### Examples 13 to 24 and Comparative Examples 4 to 6

### Preparation of Optical Recording Media

A first substrate was prepared by subjecting one side of a glass sheet 0.5 mm in thickness to an antireflective treatment to impart a reflectance of 0.1 percent for perpendicularly incident light with the wavelength of 405 nm.

A second substrate was prepared by subjecting one side of a glass sheet 0.5 mm in thickness to an aluminum vapor deposition treatment to impart a reflectance of 90 percent for perpendicularly incident light with the wavelength of 405 nm.

A transparent polyethylene terephthalate sheet 500 micrometers in thickness was provided as a spacer on the side of the first substrate that had not been subjected to the antireflective treatment.

The holographic recording compositions of Examples 1 to 4 and Comparative Examples 1 to 12 were each separately placed on first substrates, the aluminum vapor deposited surface of the second substrates were stacked on the holographic recording composition in such a manner that air was not entrained, and the first and second substrates were bonded through the spacer. Subsequently, Examples 13 to 24 and Comparative Examples 4 to 6 were left for 6 hours at 80° C. to prepare various optical recording media (holographic recording media). The thickness of the recording layers formed was 200 micrometers in all media prepared.

### <Recording in the Optical Recording Medium and Evaluation>

(1) Measurement of Recording Sensitivity

Employing a hologram recording and reproduction tester, a series of multiplexed holograms was written into the various optical recording media that had been prepared at a spot recording diameter of 200 micrometers at the focal position of the recording hologram, and the sensitivity (recording energy) was measured as follows.

### —Sensitivity Measurement—

The beam energy during recording (mJ/cm²) was varied and the change in the error rate (BER: bit error rate) of the reproduced signal was measured. Normally, there is such a tendency that the luminance of the reproduced signal increases and the BER of the reproduced signal gradually drops with an increase in the irradiated light energy. In the measurement, the lowest light energy at which a fairly good reproduced image (BER<10⁻³) was obtained was adopted as the recording sensitivity of the holographic recording medium. The wavelength of the informing light and reference light for recording as well as the wavelength of the reproduction light were 405 nm.

(2) Measurement of Recording Capacity by Planar Wave Tester

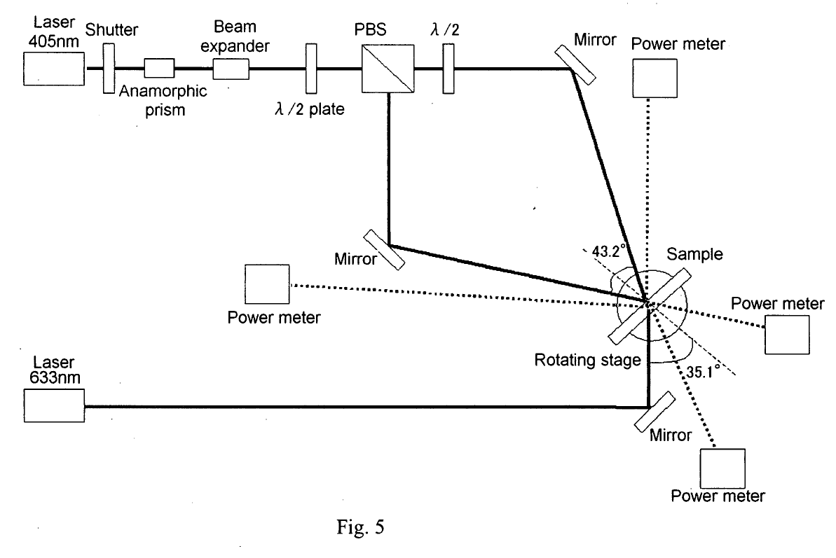

FIG. 5 shows a schematic of the optical system of a planar wave recording tester. A “Littrow” blue laser made by SONY (wavelength: 405 nm) was employed as the recording light source and an He—Ne laser (wavelength: 633 nm) that was not absorbed by the medium was employed as the probe light source. The luminous energy of the recording light source was 4 [mW] with the informing light and reference light combined. The luminous energy of the probe light source was 5 [mW]. The crossing angle of the informing light and the reference light was 43.2° (grating interval: 550 nm). The angle of incidence of the probe light—the angle at which the Bragg condition was satisfied—was 35.1°. A recording spot diameter of 6 mm was employed. The dynamic range of the storage capacity is denoted by an index referred to as “M#”. The recording capacity of each of the optical recording media of Example 13 to 24 and Comparative Examples 4 to 6 was measured with the above-described optical system. The measurement is described below.

Adopting a diffraction efficiency of 1 to 3 percent per cycle as standard, in a manner not exceeding 10 percent, 61 multiplexed recordings were conducted at intervals of 1° from −30° to +30° until the sensitivity of the recording material almost disappeared. Fixing was conducted until absorption of the recording light source by the sample almost ceased (fixing light source: High-power UV-LED (UV-400) made by Keyence), the angular selectivity was evaluated at 0.01° intervals from −32° to +32°, and the square roots of the diffraction efficiencies ηᵢ of the peaks obtained were summed to calculate M#. Diffraction efficiency η was evaluated as set forth below. The results are given in Table 2.

η=diffracted light/(diffracted light+transmitted light)×100 M#=Σ√ηᵢ

(3) Measurement of Transmittance T

The transmittance at a wavelength of 405 nm was measured with a UV-3600 (made by Shimadzu Corporation) for each of the optical recording media prepared in Examples 13 to 24 and Comparative Examples 4 to 6.

(4) Molar Absorbance Coefficient

Each of the recording monomers contained in the holographic recording compositions prepared in Examples 1 to 12 and Comparative Examples 1 to 3 was dissolved in methylene chloride to a concentration of 5×10⁻⁵ mol/L, the absorption spectrum of each solution prepared was measured with a UV-3600 (made by Shimadzu Corporation), and the absorption at 405 nm was measured. The molar absorbance coefficient was calculated from the absorbance thus measured. The results are given in Table 2.

TABLE 2

| | | | Record- | Transmittance | ε |
|---|---|---|---|---|---|
| | Holographic | Recording | ing | T of the | at 405 nm |
| | recording | sensitivity | capacity | medium at | (mole · l · |
| | composition | (mJ/cm²) | (M#) | 405 nm (%) | cm⁻¹) |
| Ex. 13 | Ex. 1 | 51 | 11.9 | 85.1 | ~0 |
| Ex. 14 | Ex. 2 | 52 | 11.8 | 85.2 | ~0 |
| Ex. 15 | Ex. 3 | 58 | 10.7 | 85.6 | ~0 |
| Ex. 16 | Ex. 4 | 32 | 13.5 | 88.1 | ~0 |
| Ex. 17 | Ex. 5 | 34 | 13.1 | 88.3 | ~0 |
| Ex. 18 | Ex. 6 | 42 | 11.9 | 88.7 | ~0 |
| Ex. 19 | Ex. 7 | 30 | 13.8 | 88.0 | ~0 |
| Ex. 20 | Ex. 8 | 31 | 13.6 | 88.1 | ~0 |
| Ex. 21 | Ex. 9 | 37 | 12.8 | 88.3 | ~0 |
| Ex. 22 | Ex. 10 | 37 | 13.0 | 88.3 | ~0 |
| Ex. 23 | Ex. 11 | 36 | 13.7 | 88.0 | ~0 |
| Ex. 24 | Ex. 12 | 36 | 13.5 | 88.2 | ~0 |
| Comp. | Comp. | 80 | 9.0 | 85.3 | ~0 |
| Ex. 4 | Ex. 1 | | | | |
| Comp. | Comp. | — | — | 0.0 | 36100 |
| Ex. 5 | Ex. 2 | | | | |
| Comp. | Comp. | — | — | 0.0 | 45200 |
| Ex. 6 | Ex. 3 | | | | |

The results of Table 2 show that the optical recording media of Examples 13 to 24, in which the holographic recording compositions of Examples 1 to 12 were employed, all had better recording sensitivity and greater recording capacity than the optical recording media of Comparative Examples 4 to 6, in which the holographic recording compositions of Comparative Examples 1 to 3 were employed. Recording and reproduction were impossible with Comparative Examples 5 and 6, which had considerable absorption at a wavelength of 405 nm.

The optical recording composition of the present invention is capable of high density recording, and is thus suitable for use in the manufacturing of various volume hologram-type optical recording media capable of high-density image recording.

Although the present invention has been described in considerable detail with regard to certain versions thereof, other versions are possible, and alterations, permutations and equivalents of the version shown will become apparent to those skilled in the art upon a reading of the specification and study of the drawings. Also, the various features of the versions herein can be combined in various ways to provide additional versions of the present invention. Furthermore, certain terminology has been used for the purposes of descriptive clarity, and not to limit the present invention. Therefore, any appended claims should not be limited to the description of the preferred versions contained herein and should include all such alterations, permutations, and equivalents as fall within the true spirit and scope of the present invention.

Having now fully described this invention, it will be understood to those of ordinary skill in the art that the methods of the present invention can be carried out with a wide and equivalent range of conditions, formulations, and other parameters without departing from the scope of the invention or any embodiments thereof.

All patents and publications cited herein are hereby fully incorporated by reference in their entirety. The citation of any publication is for its disclosure prior to the filing date and should not be construed as an admission that such publication is prior art or that the present invention is not entitled to antedate such publication by virtue of prior invention.

Unless otherwise stated, a reference to a compound or component includes the compound or component by itself, as well as in combination with other compounds or components, such as mixtures of compounds.

As used herein, the singular forms “a,” “an,” and “the” include the plural reference unless the context clearly dictates otherwise.

Except where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not to be considered as an attempt to limit the application of the doctrine of equivalents to the scope of the claims, each numerical parameter should be construed in light of the number of significant digits and ordinary rounding conventions.

Additionally, the recitation of numerical ranges within this specification is considered to be a disclosure of all numerical values and ranges within that range. For example, if a range is from about 1 to about 50, it is deemed to include, for example, 1, 7, 34, 46.1, 23.7, or any other value or range within the range.

What is claimed is:

1. An optical recording composition comprising at least one compound denoted by general formula (1): 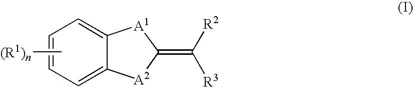
wherein, in general formula (I), each of A¹ and A² independently denotes —CR⁴R⁵—, —O—, —NR⁶—, —S—, or —C(═O)—, each of R⁴, R⁵, and R⁶ independently denotes a hydrogen atom or a substituent, R¹ denotes a substituent, n denotes an integer ranging from 0 to 4, plural substituents denoted by R¹ are identical to or different from each other when n denotes an integer of equal to or greater than 2, each of R² and R³ independently denotes a substituent having a Hammett substituent constant, σp value, of greater than 0, R² and R³ do not form a ring structure by bonding together, and at least one from among R¹, R², R³, A¹, and A² comprises at least one polymerizable group.

2. The optical recording composition according to claim 1, wherein the compound has a molar absorbance coefficient of equal to or lower than 200 mole·1·cm⁻¹ at a wavelength of 405 nm.

3. The optical recording composition according to claim 1, wherein, among A¹ and A² in general formula (I), one denotes —S— and the other denotes —NR⁶—, or one denotes —S— and the other denotes —NR⁶—.

4. The optical recording composition according to claim 1, wherein the polymerizable group is a radical polymerizable group.

5. The optical recording composition according to claim 1, wherein the compound has a maximum absorption wavelength of less than 405 nm.

6. The optical recording composition according to claim 1, further comprising at least one photo-induced polymerization initiator.

7. The optical recording composition according to claim 6, wherein the photo-induced polymerization initiator is a compound denoted by general formula (II): 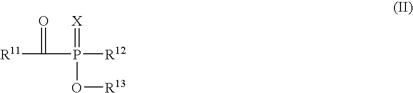
wherein, in general formula (II), each of R¹¹, R¹², and R¹³ independently denotes an alkyl group, aryl group, or heterocyclic group, and X denotes an oxygen atom or sulfur atom.

8. The optical recording composition according to claim 1, further comprising at least one polyfunctional isocyanate and polyfunctional alcohol.

9. The optical recording composition according to claim 1, which is a holographic recording composition.

10. A holographic recording medium comprising a recording layer, wherein the recording layer comprises at least one compound denoted by general formula (I): 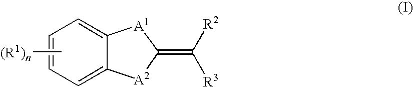
wherein, in general formula (I), each of A¹ and A² independently denotes —CR⁴R⁵—, —O—, —NR⁶—, —S—, or —C(═O)—, each of R⁴, R⁵, and R⁶ independently denotes a hydrogen atom or a substituent, R¹ denotes a substituent, n denotes an integer ranging from 0 to 4, plural substituents denoted by R¹ are identical to or different from each other when n denotes an integer of equal to or greater than 2, each of R² and R³ independently denotes a substituent having a Hammett substituent constant, σp value, of greater than 0, R² and R³ do not form a ring structure by bonding together, and at least one from among R¹, R², R³, A¹, and A² comprises at least one polymerizable group.

11. The holographic recording medium according to claim 10, wherein the compound has a molar absorbance coefficient of equal to or lower than 200 mole·1·cm⁻¹ at a wavelength of 405 nm.

12. The holographic recording medium according to claim 10, wherein, among A¹ and A² in general formula (I), one denotes —S— and the other denotes —NR⁶—, or one denotes —S— and the other denotes —NR⁶—.

13. The holographic recording medium according to claim 10, wherein the polymerizable group is a radical polymerizable group.

14. The holographic recording medium according to claim 10, wherein the compound has a maximum absorption wavelength of less than 405 nm.

15. The holographic recording medium according to claim 10, wherein the recording layer further comprises at least one photo-induced polymerization initiator.

16. The holographic recording medium according to claim 15, wherein the photo-induced polymerization initiator is a compound denoted by general formula (II): 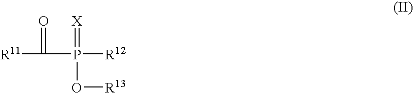
wherein, in general formula (II), each of R¹¹, R¹², and R¹³ independently denotes an alkyl group, aryl group, or heterocyclie group, and X denotes an oxygen atom or sulfur atom.

17. The holographic recording medium according to claim 10, wherein the recording layer further comprises at least one polyfunctional isocyanate and polyfinctional alcohol.
